(19) United States
(12) Patent Application Publication
(10) Pub. No.: US 2016/0154927 A1
(43) Pub. Date: Jun. 2, 2016

(54) IN SILICO METHOD TO IDENTIFY THE IMPORTANT BIOMARKERS AND COMBINATORIAL ONCOPROTEINS IN TARGET BASED CANCER THERAPY

(71) Applicant: COUNCIL OF SCIENTIFIC & INDUSTRIAL RESEARCH, Rafi Marg, New Delhi (IN)

(72) Inventors: RAMRUP SARKAR, PUNE, MAHARASHTRA (IN); SAIKAT CHOWDHURY, PUNE, MAHARASHTRA (IN)

(73) Assignee: Council of Scientific & Industrial Research, New Delhi (IN)

(21) Appl. No.: 14/906,109

(22) PCT Filed: Jul. 21, 2014
(86) PCT No.: PCT/IN2014/000478

§ 371 Date: Jan. 19, 2016

(30) Foreign Application Priority Data
Jul. 21, 2013 (IN) 1231/DEL/2013

(51) Int. Cl. G06F 19/12; C40B 30/02

(52) U.S. Cl. CPC G06F 19/12; C40B 30/02

(57) ABSTRACT

The invention is directed to in silico method to identify novel combinatorial oncoproteins that inhibit hedgehog pathway activity in various cancer cell lines required for the treatment of cancer. The invention in particular relates to in silico method to identify combinatorial oncoproteins as potential drug targets in the treatment of Glioma, Colon and Pancreatic Cancer.

### FIELD OF INVENTION

The invention is directed to in silico method to identify novel combinatorial oncoproteins that inhibit hedgehog pathway activity in various cancer cell lines required for the treatment of cancer. The invention in particular relates to in silico method to identify combinatorial oncoproteins as potential drug targets in the treatment of Glioma, Colon and Pancreatic Cancer.

### BACKGROUND OF THE INVENTION

One of the major challenges in cancer research is the identification of important biomarker proteins in the early stages of cancer diagnosis and followed by the recognition of suitable target oncoproteins to provide better therapeutic strategy. Proper identification of such target oncoproteins is utmost important for further diagnosis and treatment in cancer therapy. However, the identification of such proper target oncoproteins, which is to be monitored in the cancer pathology is still a major problem in this filed. Identification of such potential target oncoproteins requires the proper understanding of the cancer progression mechanism in cellular, molecular or genetic scale.

Hence, in order to design new therapeutic strategies for such diseases, a comprehensive study of signaling pathways for exploring these pathological manifestations, its relation with various diseases and to identify a single or combination of individual molecules that govern various different system behaviours or malfunctions is therefore essential.

Several concerted efforts are being made to dissect different signaling pathways, such as MAPK, Apoptosis, mTOR etc. and the related molecular mechanisms that control the cancer development of a cell or tissue in an organism. Among different signaling pathways, Hedgehog is of great biological relevance as it is strongly implicated in cancer development.

Hedgehog is an evolutionarily conserved developmental pathway, widely implicated in controlling various cellular responses such as cellular proliferation, stem cell renewal in human and other organisms, through external stimuli. Aberrant activation of the molecules in this pathway and its cross talk with other signaling pathways can cause various types of human cancers such as Glioma, Colon, and Pancreatic cancer. Hence, targeting this pathway in cancer therapy has become indispensable. However, complexity of the molecular interactions, complex regulations by extra- and intracellular proteins, cross talks with other pathways and non-availability of detail pathway information pose a major challenge to understand the regulatory activity of this pathway. Hence, using available drugs and targeting an individual protein in this pathway has not always proved useful to prevent its malfunction in a cancer situation.

The role of few important proteins have been identified in this pathway, such as PTCH1 (Patched receptors), SMO (Smoothened), GLI etc., which are mainly responsible for the malfunctioning of this pathway in various types of cancers. Follow-up studies by several research groups have developed therapeutic strategies to inhibit the actions of these proteins, in various cancers, but none of them have achieved complete success to cure a particular cancer that is caused by abnormal activation of the Hedgehog pathway.

Further, several attempts have been made to study the Hedgehog signaling pathway from experimental as well as theoretical and computational perspective. Dillon et al. [1]proposed reaction-diffusion kinetic models of Hedgehog signaling pathway to study Patched-Smoothened interaction and function of SHH as a long range morphogen. A dynamic model based approach to analyze the signal transduction and transport mechanism of Sonic Hedgehog to study tissue patterning has also been done successfully [2]. All these studies are based on either Ordinary Differential Equation (ODE) or Partial Differential Equations (PDE), the success of which immensely depended on reliable kinetic constants and initial concentrations, and hence requires substantial data availability of which is itself a much bigger challenge in reality. Since these are also computationally intensive, hence study of kinetic models for large network becomes challenging and difficult.

A current review by Li et al. [3], underscores the importance of combinatorial drug targets to shut off Hedgehog signaling for cancer treatment. For example, it is known that activation of cytoplasmic GLI (zinc finger transcription factor) which initiate the activity of this pathway could be regulated in two ways: (i) the ligand dependent way in which extracellular response i.e. hedgehog ligands interact with receptor proteins PTCH1/PTCH2 and activates G-coupled protein SMO, and (ii) the malfunction of the other proteins that are present in the cytoplasm which inhibit or activate its activity in the absence of hedgehog ligands. Unfortunately, till now most of the studies have mainly focused to develop a drug that will only inhibit the GLI activation, caused by the ligand dependent way. In this case most of the study is only directed to identify the drug molecule that could suppress either PTCH1 or SMO in the membrane [4-7]. These drugs, such as Cyclopamine, Vismodegib etc. are only effective when a cancer cell with excessive Hedgehog pathway activation, is encountering over-expressed hedgehog ligands (SHH, IHH or DHH) or has mutated PTCH1 or SMO in membrane. Therefore, it is clear that administration of the above mentioned drugs may not be able to cure the cancers caused by some other intracellular proteins apart from sole mutation in PTCH1 and SMO.

Further, literature reveals proteins SMO, GLI1 or GLI2 mRNA in SHH pathway responsible for glioblastoma. Article titled ‘Sonic Hedgehog/GLI1 signalling pathway inhibition restricts cell migration and invasion in human gliomas' by Wang K, [(2010) Neurol Res. 32(9):975-80]. GLI1 as key Hedgehog pathway target which is highly expressed in grade IV glioma is known in the art. Cyclopamine-Mediated Hedgehog Pathway Inhibition Depletes Stem-Like Cancer Cells in Glioblastoma’ by Eli E. et. al [(2007), STEM CELLS 25:2524-2533].

It is therefore necessary to identify alternative and multiple drug targets in this pathway. In doing so, it is imperative to study in detail interactions of the signaling proteins in this pathway and identify minimal combinations of proteins as potential drug targets in the treatment of cancer particularly for Glioma, Colon and Pancreatic cancer conditions.

Qualitative modeling approach is observed to be much more amenable to model larger biological networks than quantitative ODE approaches. In contrast to highly specific ODE based models, logic based models can model interactions between a large number of species and can be used to train, validate and generate predictions from a model [8]. This also enables the understanding of the essence of how a system functions at a larger scale before proceeding to account for the kinetic information. Previous attempts used Boolean logic to model developmental pathways for the topological study of interactions that enable prediction of patterning in Drosophila melanogaster [9], and exploration of the effect of transient perturbations on development of wild type pattern for the segment polarity network [10].

Hitherto, though diverse approaches for qualitative and quantitative methods and modelling of signaling pathways have been used to answer several biological questions in signaling systems, their, use is primarily dependent on the availability of pre-existing data and type of biological questions to be answered. Further, present inventors observed that the available databases and literature may help in understanding the mechanisms of hedgehog pathway in human cell, there are still some variations in the number of molecules and interactions, that in certain cases the information on the molecules and interactions are missing, there are other experimental studies known but the databases are not updated which pose a challenging problem to get a general structure of this signaling network and impacts the treatment of cancers including Glioma, Colon and Pancreatic cancers.

### SUMMARY OF THE INVENTION

In accordance with the above, the present invention provides an in silico method for identification of novel combinatorial oncoproteins, as potential drug targets, that inhibit hedgehog pathway activity useful in treatment of Glioma, Colon and Pancreatic cancers. The invention provides a comprehensive hedgehog pathway, that can help to identify several proteins hitherto unknown and their interactions for treatment of Glioma, Colon and Pancreatic cancer.

In an aspect, the present invention provides novel combinatorial f proteins comprising SMO, HFU, ULK3 and RAS as potential drug targets in Hedgehog pathway to control or treat colon cancer.

In another aspect, the present invention provides combinatiorial oncoproteins comprising SMO, GLI1, GLI2, as potential drug targets in Hedgehog pathway to control or treat Glioma Grade IV cell line.

In yet another aspect, the present invention provides novel biomarkers/combination of proteins comprising SMO, HFU, ULK3, RAS and ERK12 as potential drug targets in Hedgehog pathway to control or treat pancreatic cancer.

In another aspect, the present invention provides a comprehensive hedgehog pathway comprising of 57 proteins (52 core proteins and 5 cross talk protein molecules from other pathways), 6 cellular or phenotypic expression and 96 hyper-interactions (using the information from different sources as disclosed in Table 1 & 2). This comprehensive hedgehog pathway provides an extensive and up to date signaling map that can serve both experimental as well as theoretical biology communities.

In another aspect, the present invention discloses an in silico method for identification of novel combination of oncoproteins in the hedgehog signalling pathway as potential drug targets to control or treat cancer in a subject where, the method of identification of target proteins is based on Graph theoretic and logical analysis.

Accordingly, the present inventors simulated the logical models of Hedgehog pathway for normal scenario as well as for three different types of cancer i.e. Glioma, Colon and Pancreatic cancer in Cell Net Analyzer [23] to identify the proteins that are involved in the abnormal activation of hedgehog pathway resulting in these three types of cancers.

The modulation of the combination proteins identified in the hedgehog pathway as potential drug targets results in the down regulation of the output proteins associated with glioma, colon and pancreatic cancer such as JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA with the phenotypic outcomes or cellular responses (like Cell proliferation, Cell cycle progression and Endothelial to Mesenchymal transition etc.) and also with three other important pathways, WNT, NOTCH and Anti-Apoptosis.

The inclusion of non-core hedgehog pathway proteins such as ERK12, RAS, TWIST, FAS, NOTCH1, in the reconstructed map expresses the regulation or cross talks of hedgehog pathway with other molecules from different signaling pathways like WNT, NOTCH, MAPK etc.

### BRIEF DESCRIPTION OF THE DRAWINGS

FIG. 1 depicts Venn diagram represents a comparative view of number of proteins in current model with existing major databases, KEGG, BIOCARTA, GENE GO, NETPATH and PATHWAY CENTRAL, considered to reconstruct the Hedgehog pathway diagram.

The overlapping regions between two circles (i.e. two databases or anyone of the database and the instant model) represent proteins common to both the databases. The large non-overlapping area shown by the instant model signifies information of the large number of proteins which are not found in any of the above mentioned databases and are taken from other literature sources.

FIG. 2 depicts All pairs shortest paths of the proteins of Hedgehog signalling network. The values of shortest path(s) between two proteins in the Hedgehog signalling network is presented with the name of the proteins arranged in both row and column wise. Different colors are used to distinguish the different values of shortest path. White cells represent zero value or no shortest path. The lower part (i.e. from CYCLIN_B to CYCLIN_D2) corresponds to the Output proteins of Hedgehog pathway and hence there are no connections of these proteins with the remaining proteins in network (FIG. 5).

FIG. 3 depicts Probability distributions of the Shortest paths of Hedgehog signalling network.

The X-axis represents the value of the shortest paths from 1 to 8 and Y-axis represents the probability of deriving a particular shortest path in the network. The shortest path ‘3’ has highest probability in the distribution and the average shortest path is calculated as 3.581.

FIG. 4 depicts the newly constructed Hedgehog Pathway.

The total 57 proteins of this network are allocated into four main regions with different color codes: Extracellular and Membrane; Cytoplasm; Nucleus; and Output. The output proteins are linked with various cellular responses (cross talk with other pathways or phenotypic expressions) with black dotted arrow. The green and red arrows indicate Activation/Production and Inhibition process respectively. The black arrows indicate the nuclear translocation process.

FIG. 5 depicts Network picture of Hedgehog signaling pathway.

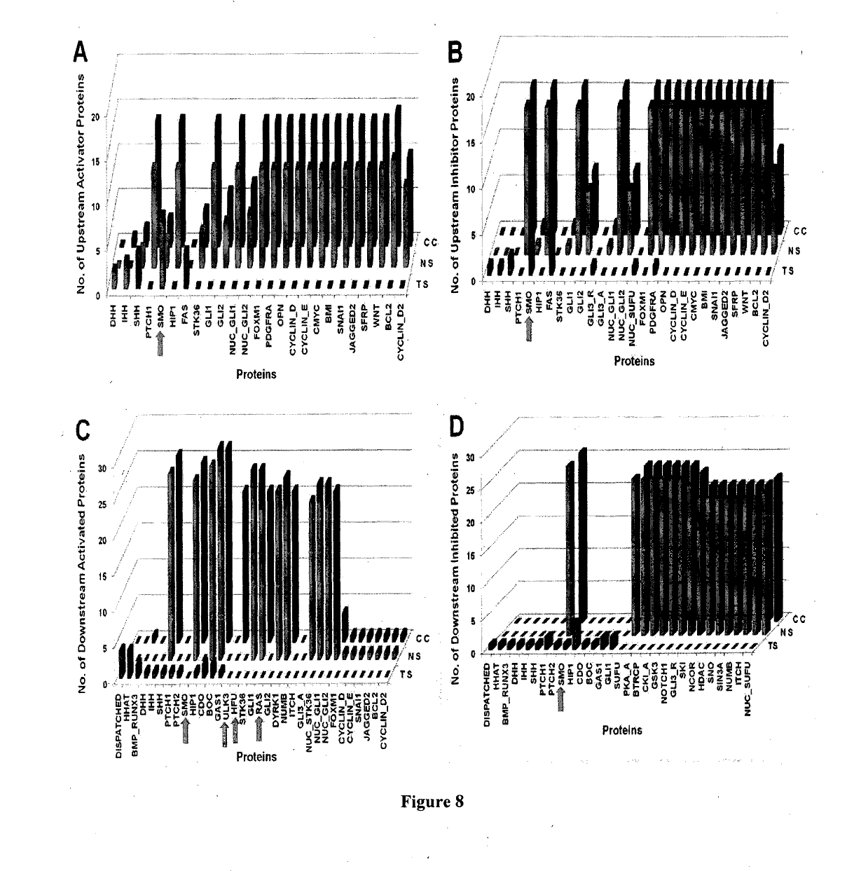

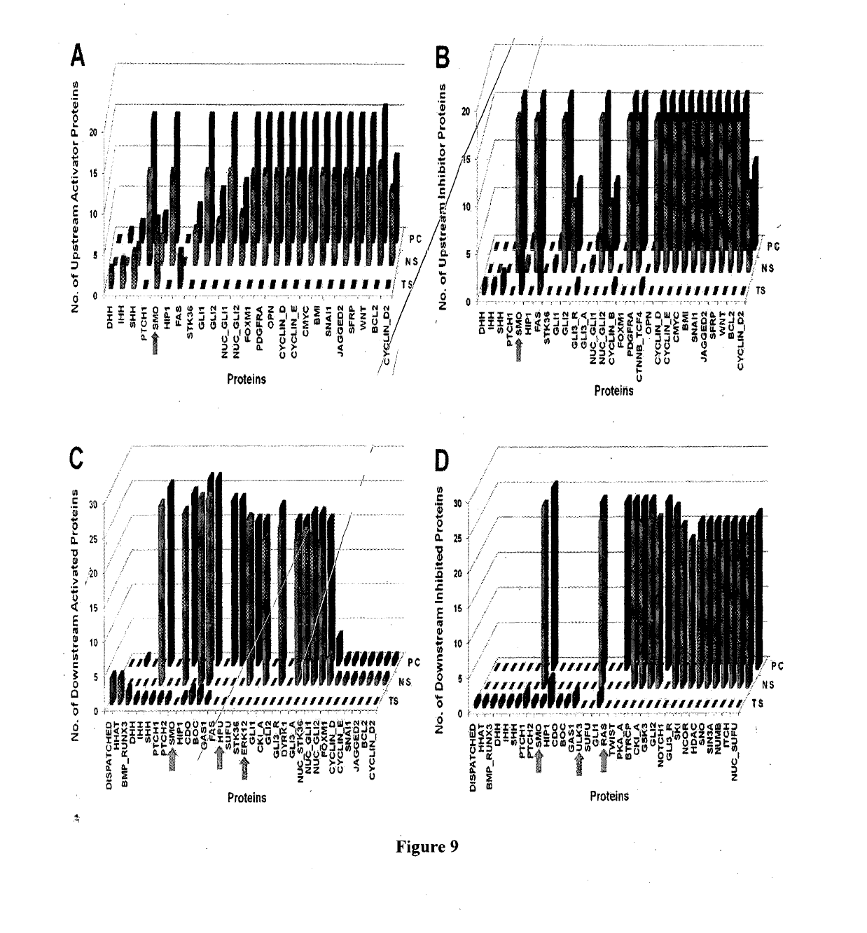

The colored circles represent the nodes or proteins of the pathway and the black arrows indicate an edge or connection between two nodes of the network. The nodes are colored according to their sub cellular locations in the cell (FIG. 4) and are divided into four regions: Extracellular and membrane, Cytoplasm, Nuclear and Output proteins respectively. The size of the nodes is assigned according to their total number of connections or degree. and is followed by the name of the proteins. The “Bow-Tie” structure of the Hedgehog signaling network is easily visible, where the signals are converge towards GLI1 or GLI2 and diverge to its subsequent steps. The size the node GLI1 is biggest as it has highest degree among all other proteins, in the network.

FIG. 6 depicts Parameter values from Connectivity and Centrality Analysis indicated by the heat map of the values of the parameters.

The names of the proteins or nodes are arranged row wise (Y-axis) according to the position of their corresponding region (FIG. 4). The parameter values are arranged column wise (X-axis) in the heat map. FIG. 6(A) represents the heat map of the values of the parameters values used in connectivity analysis: IN-DEGREE, OUT-DEGREE and TOTAL DEGREE of each protein. High IN-DEGREE value of GLI1, PTCH1, HHIP and SHH indicates their higher number of up-regulation by the other proteins in the network. High OUT-DEGREE value of several nuclear proteins (e.g. DYRK1, NUMB, NUC_GLI1, NUC_SUFU, NUC_STK36 etc) refers their ability to regulate other proteins in HH network. In case of total degree, GLI1, GLI2 and NUC_GLI1 have significant highest value. It refers that these two proteins are mostly connected to the other proteins in the network. FIG. 6 (B) represents heat map of the individual centrality score of each protein of Hedgehog map. The Centrality measurement parameters used in this analysis were Eigenvector (EC), Betweenness (BC) and Closeness (CC) centrality. It is observed that GLI1 has the highest value for each parameter score. Subsequently, PTCH1, PTCH2, HHIP, STK36, NUC_GLI1, NUC_GLI2 etc. are also showing significant value for each individual centrality score.

FIG. 7 depicts comparison between Normal, Cancer and Perturbed scenarios for Glioma [TS: Treated Scenario; NS: Normal Scenario; GS: Glioma Scenario].

The arrow heads indicate the minimal combination of proteins inhibited in the drug treated perturbation analysis. (A) represents number of Upstream activator proteins (Y-axis) activating the proteins (X-axis) representing significant variations. Compared to the normal scenario, proteins, SHH, SMO, GLI1, GLI2, and output proteins BMI, SNAI1, BCL2 and Cyclins, are activated by maximum number of upstream activator proteins. On drug treated perturbation of SMO, GLI1 and GLI2, the number of activators of the output proteins become zero. (B) represents number of Upstream inhibitory proteins (Y-axis) inhibiting the proteins (X-axis) representing significant variations. The numbers of upstream inhibitor proteins in normal versus Glioma scenario remain same. Similar perturbation results are observed in (A). (C) Represents number of Downstream proteins (Y-axis) activated by the proteins (X-axis) representing significant variations. The number of downstream proteins activated in normal versus Glioma remains same. On perturbation of SMO, GLI1 and GLI2, the number of downstream proteins activated by these proteins is reduced to zero. (D) represents number of Downstream proteins (Y-axis) inhibited by the proteins (X-axis) representing significant variations. The number of downstream proteins inhibited in normal versus Glioma remains the same.

FIG. 8 depicts comparison between Normal, Cancer and Perturbed scenarios for Colon Cancer

TS: Treated Scenario; NS: Normal Scenario; CC: Colon Cancer Scenario. The arrow heads indicate the minimal combination of proteins which was inhibited in the drug treated perturbation analysis. (A) Represents number of Upstream activator proteins (Y-axis) activating the proteins (X-axis) representing significant variations. Compared to the normal scenario, proteins, SHH, IHH, SMO, GLI1, GLI2 and output proteins BMI, SNAI1, BCL2 and Cyclins are activated by maximum number of upstream activator proteins. On drug treated perturbation of SMO, HFU, ULK3 and RAS, the number of activators of the output proteins become zero. (B) Represents number of upstream inhibitory proteins (Y-axis) inhibiting the proteins (X-axis) representing significant variations. The numbers of upstream inhibitor proteins in normal versus Colon cancer scenario remain same. Similar perturbation results are observed as in (A). (C) Represents number of downstream proteins (Y-axis) activated by the proteins (X-axis) representing significant variations. The number of downstream proteins activated in normal versus Colon cancer scenario remains same. On perturbation of SMO, HFU, ULK3 and RAS, the number of downstream proteins activated by these proteins is reduced to zero. (D) Represents number of downstream proteins (Y-axis) inhibited by the proteins (X-axis) representing significant variations. The numbers of downstream proteins inhibited in normal versus Colon cancer scenario remain same.

FIG. 9 depicts comparison between Normal, Cancer and Perturbed scenarios for Pancreatic Cancer

[TS: Treated Scenario; NS: Normal Scenario; PC: Pancreatic Cancer Scenario]. The arrow heads indicate the minimal combination of proteins which was inhibited in the drug treated perturbation analysis. (A) Represents number of Upstream activator proteins (Y-axis) activating the proteins (X-axis) representing significant variations. Compared to the normal scenario, proteins, SHH, IHH, SMO, GLI1, GLI2 and output proteins BMI, SNAI1, BCL2 and Cyclins are activated by maximum number of upstream activator proteins. On drug treated perturbation of SMO, HFU, ULK3, RAS and ERK12, the number of activators of the output proteins become zero. (B) Represents number of upstream inhibitory proteins (Y-axis) inhibiting the proteins (X-axis) representing significant variations. The numbers of upstream inhibitor proteins in normal versus Pancreatic cancer scenario remain same. Similar perturbation results are observed as in (A). (C) Represents number of downstream proteins (Y-axis) activated by the proteins (X-axis) representing significant variations. The numbers of downstream proteins activated in normal versus Pancreatic Cancer remain same. On perturbation of SMO, HFU, ULK3, RAS and ERK12, the number of downstream proteins activated by these proteins is reduced to zero. (D) Represents number of downstream proteins (Y-axis) inhibited by the proteins (X-axis) representing significant variations. The numbers of downstream proteins inhibited in normal versus Pancreatic cancer scenario remain same.

FIG. 10 depicts comparison of protein expression levels observed in experiment and model simulation for different cancer situations EXP: Experimental data observed from published literatures; SIM1: Simulation 1 performed using the expressions data from Table 4; SIM2: Simulation 2 performed using the expressions levels observed from experimental data. (A) Represents the comparisons of the expressions of hedgehog pathway proteins found in the experimental data (EXP) of Glioma Grade IV cell line [24] and in the corresponding simulation (SIM1 and SIM2) data from logical model. (B) Represents the comparisons of the expressions of hedgehog pathway proteins found in the experimental data (EXP) of Colon cancer cell line [25] and in the corresponding simulation (SIM1 and SIM2) data from logical model. (C) Represents the comparisons of the expressions of hedgehog pathway proteins found in the experimental data (EXP) of Pancreatic cancer cell line [26] and in the corresponding simulation (SIM1 and SIM2) data from logical model.

FIG. 11 depicts Protein expression levels observed in SMO inhibition and Treatment Scenarios (Combination therapy) for different cancers First columns of (A), (B) and (C) represent the expressions of the proteins found after inhibiting SMO in Glioma, Colon and Pancreatic cancer models, respectively. Second columns of (A) represents the expressions of the proteins observed in the treatment scenario by perturbing SMO, GLI1 and GLI2 in combination in the same Glioma model; (B) represents the expressions of the proteins in the treatment scenario by perturbing SMO, HFU, ULK3, and RAS in combination in the same Colon cancer model; (C) represents the expressions of the proteins in the treatment scenario by perturbing SMO, HFU, ULK3, ERK12, and RAS in combination in the same Pancreatic cancer model. (D), (E) and (F) represent the identified alternative pathways (shown by solid arrows) that remain active even after the inhibition of SMO in membrane (pathway shown by broken arrows) by its inhibitor molecule (i.e. Cyclopamine, Vismodegib etc.) in Glioma, Colon and Pancreatic cancer scenarios, respectively.

### DETAILED DESCRIPTION OF THE INVENTION

While the invention is susceptible to various modifications and/or alternative processes and/or compositions, specific embodiment thereof has been shown by way of example in the drawings/figures and tables and will be described in detail below. It should be understood, however that it is not intended to limit the invention to the particular processes and/or compositions disclosed, but on the contrary, the invention is to cover all modifications, equivalents, and alternative falling within the spirit and the scope of the invention as defined by the appended claims.

The graphs, tables, formulas and protocols have been represented where appropriate by conventional representations in the drawings, showing only those specific details that are pertinent to understanding the embodiments of the present invention so as not to obscure the disclosure with details that will be readily apparent to those of ordinary skill in the art having benefit of the description herein.

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention.

The terms “comprises”, “comprising”, or any other variations thereof, are intended to cover a non-exclusive inclusion, such that one or more processes or composition/s or systems or methods proceeded by “comprises . . . a” does not, without more constraints, preclude the existence of other processes, sub-processes, composition, sub-compositions, minor or major compositions or other elements or other structures or additional processes or compositions or additional elements or additional features or additional characteristics or additional attributes.

### DEFINITIONS

For the purposes of this invention, the following terms will have the meaning as specified therein:

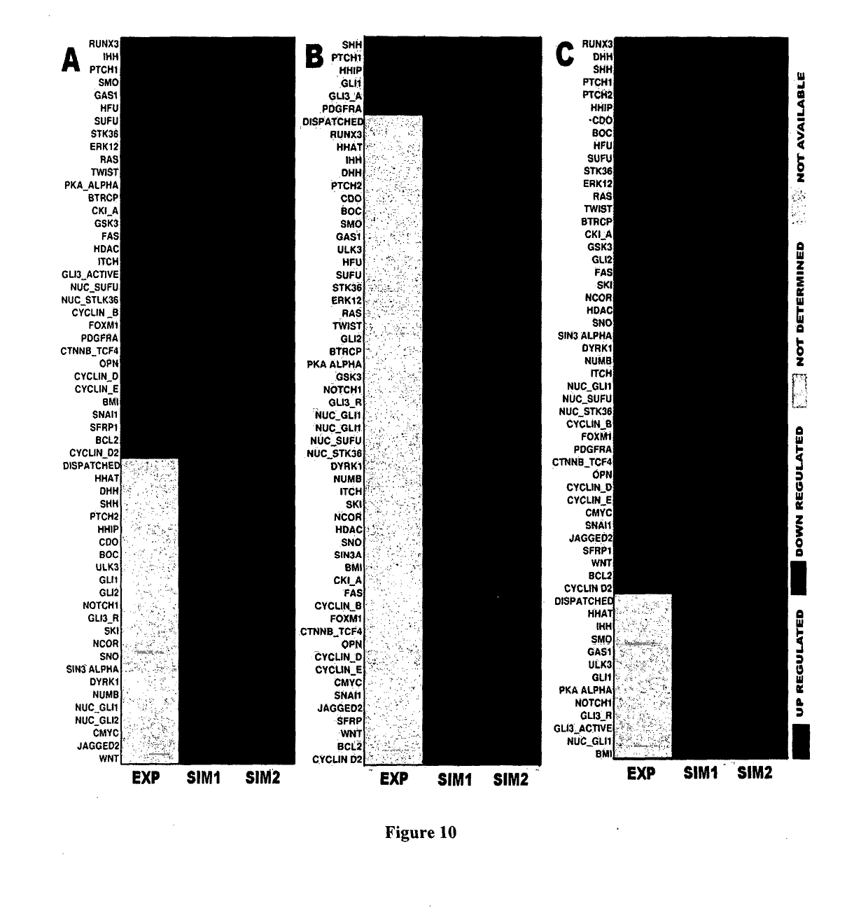

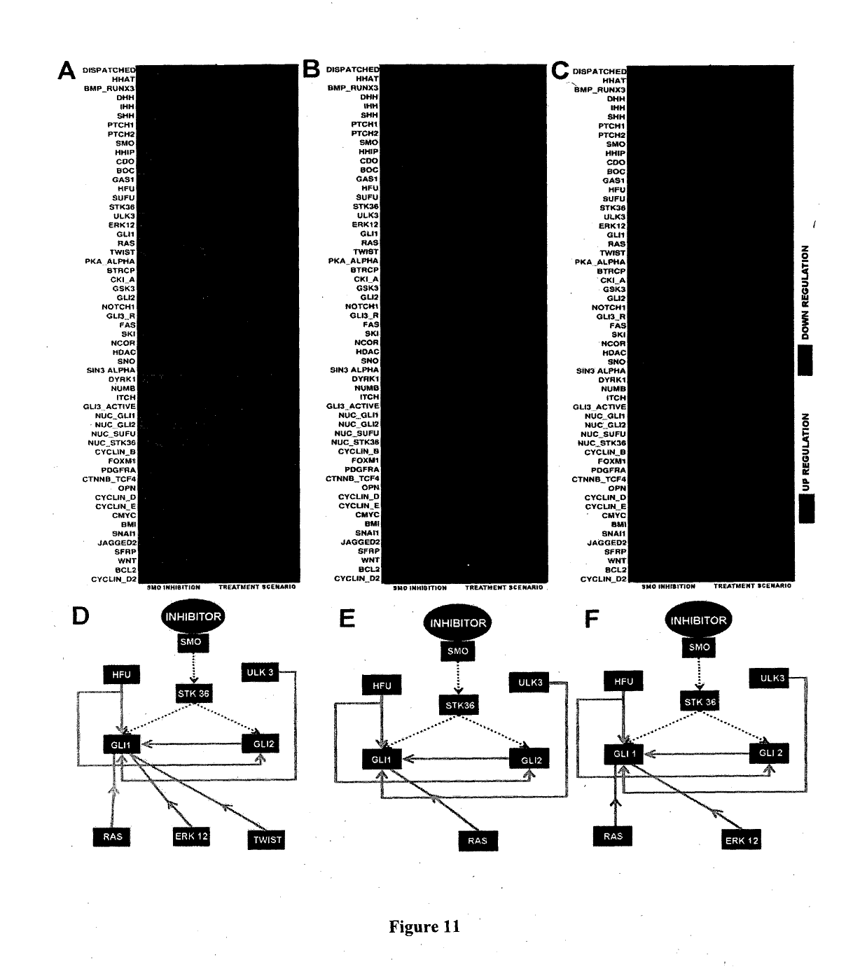

In-Degree (Kin): It refers the total number of nodes (activations or inhibitions) that are directly acting on a particular node in the network.
Out-Degree (Kout): The total number of interactions (activations or inhibitions) that are acting by a particular node on the other nodes in the network.
Degree (Ki): It refers the total number of in-degree and out-degree of a particular node.
Eigenvector centrality: It refers that a node in a network will be more central if it isconnected to many central nodes in the network.
Betweeness centrality: It is the ratio of the number of shortest paths that pass through the node to the total number of shortest paths of all the nodes to all the other nodes. It signifies that how a node is important in the shortest paths of all the other nodes of the network.
Closeness centrality of a node: It is defined as the inverse of sum of the total length of the distances or shortest paths of that node to the other nodes. Therefore higher closeness centrality of a node implies the lower length of shortest paths to the all other nodes in the network and signifies how close a node is situated from the other nodes in the network.
Shortest Path (Lij): It refers the minimum number of intermediate links or connections that have to traverse from one node ‘i’ to the another node ‘j’.
Upstream Activator proteins. It defines the proteins which are present at the upstream of a protein and help to activate its expression.
Downstream Activated proteins: It defines the proteins which are present at the downstream of a protein and are activated or up regulated by the influence of that protein.
Upstream Inhibitor proteins. It defines the proteins which are present at the upstream of a protein and inhibit or down regulate its expression.
Downstream Inhibited proteins: It defines the proteins which are present at the downstream of a protein and are inhibited or down regulated by the influence of that protein.
Cancer Scenarios: Different Cancer scenarios are created by altering the logical states (“0” as “OFF” or “1” as “ON”) of the input proteins. We considered both the ‘gain-of-function’ (“ON” or “1”) states of the oncogenic proteins like RAS, ERK12, TWIST etc., and the “loss of functions” (“OFF” or “0”) of few tumor suppressor proteins like GAS1, SUFU, NUMB, SNO while creating different cancer scenarios in the logical simulation (See Table 4). There were few exceptions in our model while considering the input proteins for three cancer scenarios (Glioma, Colon and Pancreatic) with respect to the normal pathway scenario. In that case, we considered the three ligands SHH, IHH and DHH as inputs, though they had the upstream connections. In order to simulate their over expression effects in the cancer scenarios, we kept their constant active state as 1 or ON throughout the simulations.
Norma Scenarios. This is the normal expression profile of the proteins in the canonical hedgehog pathway as reported in different literatures and sources. In our simulation we have considered their logical state values according to the normal pathway expression (see Table 3). In normal scenario, we considered the expression or logical state of the three ligands SHH, IHH and DHH according to the state of their upstream activators (e.g. BMP_RUNX3, CDO, BOC etc.).
Input and Output protein: In order to construct a logical model for hedgehog signaling pathway, at first we considered the input and output nodes. Mainly the proteins, which did not have upstream connection in the reconstructed pathway map (FIGS. 4 and 5), were considered as Input proteins. Similarly, the proteins, which were the downstream effectors of input proteins, were considered as Output proteins in our model. The names of the input and output proteins was provided in Table 3 with proper documentation. The logical states of the input and output proteins are given in Table 4, where the logical states of the input proteins considered from various literature sources, EBI-ArrayExpress Atlas and also from various signaling and cancer databases (See Table 1), for further simulation to derive the state of the output proteins. We included the connections of the output proteins JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA with the phenotypic outcomes or cellular responses (like Cell proliferation, Cell cycle progression and Endothelial to Mesenchymal Transition etc.) and also with three other important pathways, WNT, NOTCH and Anti-Apoptosis
Non-Core hedgehog pathway protein: We included ERK12, RAS, TWIST, FAS, NOTCH1, which are not the core hedgehog pathway proteins (see FIGS. 4 and 5). The inclusion of these proteins in our reconstructed map was to show the regulation or cross talks of hedgehog pathway with other molecules from different signaling pathways like WNT, NOTCH, MAPK etc. Including these non-core proteins, as far as the literature and database are concerned, this reconstructed map of Hedgehog signaling pathway represents the highest number of molecules and interactions, and is considered for further computational analysis.

Thus the present invention is directed to an in silico method to identify novel combinations of proteins and their interactions in the hedgehog pathway for cancer treatment, specifically in treatment of Glioma, Colon and Pancreatic cancers. The present invention also provides for in silico method to identify novel combinations of biomarker proteins and their interactions in the hedgehog pathway for cancer treatment, specifically in treatment of Glioma, Colon and Pancreatic cancers.

In one aspect the present invention provides to identify novel combinatorial proteins and their interactions hitherto unknown for cancer treatment by constructing a comprehensive hedgehog pathway. In yet another aspect the present invention provides to capture the molecular interactions of newly identified combinations of oncoproteins using newly constructed comprehensive hedgehog pathway by an in silico method. In another aspect the present invention provides novel therapeutic strategy by targeting combinatorial oncoproteins that inhibit hedgehog pathway activity in various cancer cell lines such as Glioma, Colon and Pancreatic cancers.

The invention provides a comprehensive hedgehog pathway, that can help to identify several proteins hitherto unknown and their interactions for new combinatory drug targets to control different cancers. Comparing the cancer scenarios with normal scenario, the present inventors have observed that minimal important combinations of proteins may be used as drug targets for in-vitro and in-vivo analysis of cancer. Thus, through Reconstruction, Graph theoretic and Boolean analysis of the pathway, the present invention aims to provide a novel combination of proteins in the Hedgehog pathway that may be used as potential drug targets for Glioma, Colon and Pancreatic cancer, where the pathway is known to become mutated.

In an embodiment, the modulation of the combination proteins identified in the hedgehog pathway as potential drug targets results in, the down regulation of the output proteins associated with glioma, colon and pancreatic cancer such as JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA with the phenotypic outcomes or cellular responses (like Cell proliferation, Cell cycle progression and Endothelial to Mesenchymal transition etc.) and also with three other important pathways, WNT, NOTCH and Anti-Apoptosis.

These out-put proteins in the instant pathway are identified as oncoproteins which thus helps to understand the working of the HH (hedgehog) pathway in controlling the major developmental procedures of a cell such as cell division, cell proliferation and crosstalks with other pathways.

Further, inclusion of non-core hedgehog pathway proteins such as ERK12, RAS, TWIST, FAS, NOTCH1 allows to display the regulation or cross talks of hedgehog pathway with other molecules from different signalling pathways like WNT, NOTCH, MAPK and other pathways.

The new comprehensive hedgehog pathway thus provides an extensive and up to date signaling map that can serve both experimental as well as theoretical biology communities.

The new comprehensive Hedgehog pathway (FIG. 4) is a master model that accounts for all the possible proteins and their interactions in different cell types across different experimental conditions. The pathway includes all the probable proteins and interactions that govern the flow of the signal, from input to intermediate to output layer.

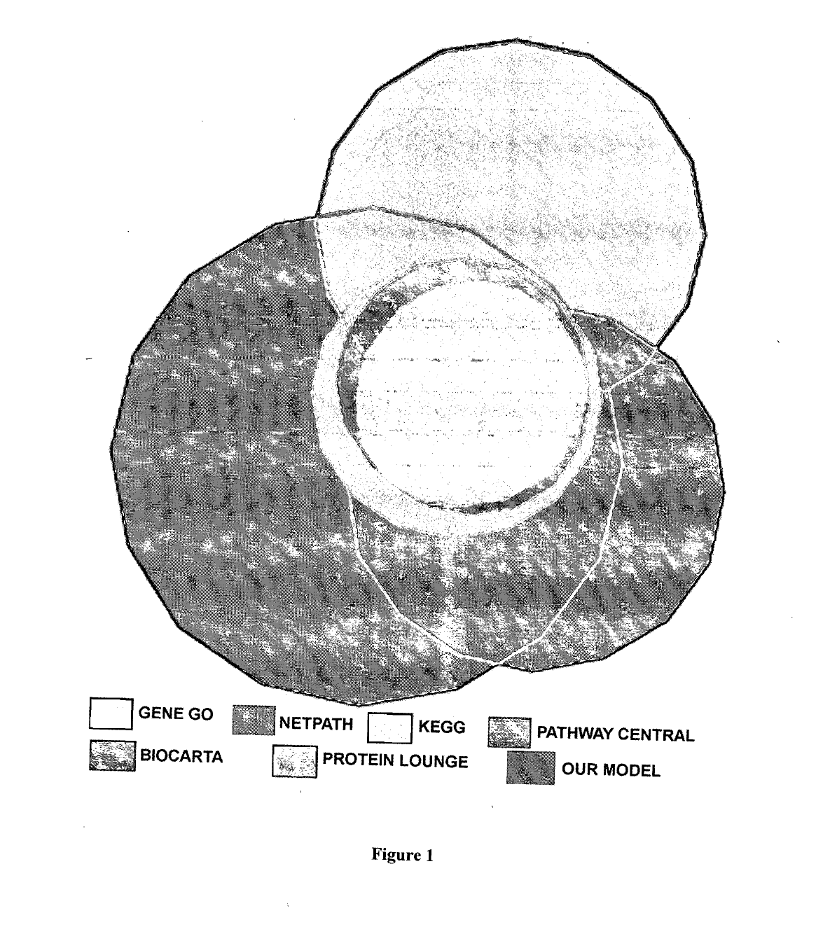

The new comprehensive Hedgehog pathway (FIG. 4) is reflected in Venn diagram (FIG. 1) constructed to compare between the number of proteins available in major database models and the proteins in the present model. The proteins identified in the instant invention are represented by the non-overlapping region in Venn diagram.

In an embodiment, the present invention provides a comprehensive hedgehog pathway comprising of 57 proteins (52 core proteins and 5 cross talk protein molecules from other pathways), 6 cellular or phenotypic expression and 96 hyper-interactions (using the information from different sources as disclosed in Table 1 & 2).

Thus, in an embodiment, the present invention discloses the novel minimal combinations of proteins which can be used as drug targets for further in-vitro and in-vivo analysis in treatment of cancer.

In an embodiment, the present invention discloses novel combinatorial proteins such as GLI1, GLI2 and SMO as potential drug targets in Hedgehog pathway to control or treat glioma.

In another embodiment, the invention provides novel combinatorial proteins such as SMO, HFU, ULK3 and RAS as potential drug targets in Hedgehog pathway to control or treat colon cancer.

In yet another embodiment, the invention provides novel combinatorial proteins such as SMO, HFU, ULK3, RAS and ERK12 as potential drug targets in Hedgehog pathway to control or treat pancreatic cancer.

In another embodiment, the present invention discloses an in silico method for identification of novel combination of oncoproteins in the hedgehog signalling pathway as potential drug targtes comprising comparing the expression of proteins, wherein the identification of target proteins to treat cancer is based on Graph theoretic and logical analysis.

Accordingly, the in silico method comprises;

(i) reconstructing a novel hedgehog pathway by collating proteins from the various databases; and
(ii) simulating the logical models of the Hedgehog pathway for normal scenario as well as for cancer scenario in Cell Net Analyzer to identify the proteins as drug targets involved in the abnormal activation of hedgehog pathway in the development of cancer.

The logical analysis of step (ii) described above further comprises;

comparing computationally the number of upstream activator proteins DHH, SHH, IHH, PTCH1, SMO, HIP1, STK36, GLI1, GLI2, GLI3_R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7A, 8A and 9A; number of downstream activated proteins of DISPATCHED, HHAT, BPM_RUNX3, SHH, DHH, IHH, PTCH1, PTCH2, SMO, HIP1, CDO, BOC, GAS1, ULK3, RAS, STK36, HFU, SUFU, TWIST, PKA_A, BTRCP, CKI_A, GSK3, NUC_GLI1, NUC_GLI2, CYCLIN_B, FOXM1, CYCLIN_D, CYCLIN_E, SNAI1, JAGGED2BCL2, CYCLIN_D2 selected from FIGS. 7C, 8C, and 9C; number of upstream inhibitor proteins of DHH, SHH, IHH, PTCH1, SMO, HIP1, STK36, GLI1, GLI2, GLI3_R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7B, 8B and 9B; and number of downstream inhibited proteins of DISPATCHED, HHAT, BPM_RUNX3, SHH, DHH, IHH, PTCH1, PTCH2, SMO, HIP1, CDO, BOC, GAS1, ULK3, RAS, STK36, HFU, SUFU, TWIST, PKA_A, BTRCP, CKI_A, GSK3, NOTCH1, GLI3_R, SKI, NCOR, HDAC, SNO, SIN3A, NUMB, ITCH, NUC_SUFU selected from FIGS. 7D, 8D and 9D of the cancer scenario with each protein of the normal scenario.
(i) identifying and extracting the proteins those number of upstream activator proteins such as SMO, HFU, ULK3, RAS and ERK12 with significant variations in cancer scenarios with respect to the normal scenario,
(ii) selecting combinations of target proteins from step (ii) for each cancer scenario and perturbing said combination of proteins in the treatment scenario; and
(iii) inhibiting the expression of the output oncoproteins of the hedgehog pathway causing cancer.

In the in silico method of identification, it is observed that the number of upstream activator proteins in the cancer scenario is greater than that of the normal scenario thereby subsequently effecting the expression of the output oncoproteins. Each target protein is assigned ‘0’ or ‘OFF’ and ‘1’ or ‘ON’ to up regulate or down regulate the expression of said protein. The output oncoproteins comprises JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA.

In an embodiment, the invention provides a comparison between normal Hedgehog scenario along with three types of cancers Glioma, Colon and Pancreatic cancer as well as their perturbed/treated scenarios respectively. The proteins in FIGS. 7, 8 and 9 are presented along X-axis and three types of scenarios i.e. Normal Scenario (NS); Cancer Scenario [Glioma (GS); Colon (CC); Pancreatic (PC)]; Treatment Scenario (TS) along the Y-axis and the number of upstream activator/inhibitor proteins (A/B) or downstream activated/inhibited (C/D) proteins along the Z-axis. Since, The instant model of ‘canonical hedgehog pathway’ or ‘normal scenario’ mimicked the expression scenarios of various proteins in a canonical hedgehog pathway of a non-malignant, non-cancerous cell line, and was used to identify the combination of proteins as target proteins for further perturbation analysis and combinatorial drug treatment scenario (TS) in Glioma, Colon and Pancreatic cancer.

Accordingly, the present inventors simulated the logical models of Hedgehog pathway for normal scenario as well as for three different types of cancer i.e. Glioma, Colon and Pancreatic cancer in Cell Net Analyzer [23] to identify the proteins that are involved in the abnormal activation of hedgehog pathway resulting in the development of these three types of cancers.

The structural analysis of instant Hedgehog signaling network indicated that protein GLI1 forms a “hub”, within the entire network and perturbation (i.e. mutation, malfunction, high or low expression etc.) of this protein or node in said Hedgehog signalling network affected the normal network function and caused several types of cancers, such as Glioma, Colon and Pancreatic cancer cell lines where GLI1 show over expression.

In addition, to transmit the activation signal after ligand binding to the PTCH1 and PTCH2 receptors, SMO was observed to become active. Due to this reason, this membrane bound receptor protein was identified as possible potential drug targetable protein in the cancer caused by Hedgehog pathway activation.

In another embodiment, the present invention provides an in silico method for selecting cancer treatment regime for a subject, specifically for Glioma, Colon and Pancreatic cancer, comprising perturbation of novel combinations of proteins of Hedgehog signalling pathway followed by suppressing the expression of onco proteins.

In an embodiment, the present invention provides an in silico method for selecting cancer treatment regime for glioma/glioma grade IV cell line in a subject comprising perturbation of a combination of proteins selected from SMO, GLI1 and GLI2 to supress the expression of GLI transcription factors and subsequently inhibiting output proteins such as JAGGED2, WNT, SFRP, CYCLIN_B, CYCLIN_D, CYCLIN_D2, CYCLIN_E, OPN, SNAI1, CMYC, BMI, BCL2, FOXM1, PDGFRA of the hedgehog pathway.

Accordingly, altering the logical states of SMO, GLI1 and GLI2 proteins from 1 (‘ON”) to 0(“OFF”) in Glioma model, resulted in suppressing the expression of various output proteins (e.g. JAGGED2, WNT, SFRP, CYCLIN_B, CYCLIN_D, CYCLIN_D2, CYCLIN_E, OPN, SNAI1, CMYC, BMI, BCL2, FOXM1, PDGFRA etc.) as well as the phenotypic expressions of the Glioma affected cell (FIG. 7). Hence, inhibition of these proteins was helpful for the therapeutic treatment of Glioma.

In another embodiment, the present invention provides an in silico method for selecting cancer treatment regime for colon cancer comprising perturbation of a combination of proteins selected from SMO, HFU, ULK3 and RAS to supress the expression of GLI transcription factors and subsequently inhibiting output proteins such as JAGGED2, WNT, SFRP, CYCLIN_B, CYCLIN_D, CYCLIN_D2, CYCLIN_E, OPN, SNAI1, CMYC, BMI, BCL2, FOXM1, PDGFRA of the hedgehog pathway.

Accordingly, altering the logical states of SMO, HFU, ULK3 and RAS from 1 (“ON”) to 0 (“OFF”) in colon cancer resulted in suppressing the expression of various output proteins such as JAGGED2, WNT, SFRP, CYCLIN_B, CYCLIN_D, CYCLIN_D2, CYCLIN_E, OPN, SNAI1, CMYC, BMI, BCL2, FOXM1, PDGFRA as well as the phenotypic expressions of the colon cancer cell. Hence, inhibition of these proteins was helpful for the therapeutic treatment of colon cancer (FIG. 8).

In yet another embodiment, the present invention provides an in silico method for selecting cancer treatment regime for pancreatic cancer comprising perturbation of a combination of proteins selected from SMO, HFU, ULK3, RAS and ERK12 to supress the expression of GLI transcription factors and subsequently inhibiting output proteins such as JAGGED2, WNT, SFRP, CYCLIN_B, CYCLIN_D, CYCLIN_D2, CYCLIN_E, OPN, SNAI1, CMYC, BMI, BCL2, FOXM1, PDGFRA of the hedgehog pathway.

Thus, altering the logical states of SMO, HFU, ULK3, RAS and ERK12 from 1 (“ON”) to 0 (“OFF”) in pancreatic cancer resulted in suppressing the expression of various output proteins JAGGED2, WNT, SFRP, CYCLIN_B, CYCLIN_D, CYCLIN_D2, CYCLIN_E, OPN, SNAI1, CMYC, BMI, BCL2, FOXM1, PDGFRA as well as the phenotypic expressions of the pancreatic cancer cell line. Hence, inhibition of these proteins was helpful for the therapeutic treatment of pancreatic cancer. (FIG. 9).

The novel combination of proteins identified in the hedgehog pathway in the instant invention can be used as target proteins to treat or cure glioma, colon and pancreatic cancer thereby inhibiting the hedgehog pathway in respective cancer scenario. Accordingly, the main embodiment of the present invention provides novel combinatorial oncoproteins comprising SMO, HFU, ULK3, RAS and ERK12 as potential drug targets in the Hedgehog pathway to control or treat cancer.

Another embodiment of the present invention provides for the combinatorial oncoproteins as described herein in the present invention wherein the cancer is colon or pancreatic cancer.

Another embodiment of the present invention provides for the combinatorial oncoproteins as described herein in the present invention, wherein the combinatorial oncoproteins are selected from SMO, HFU, ULK3, RAS as potential drug targets in the Hedgehog pathway to control or treat colon cancer.

Another embodiment of the present invention provides for the combinatorial oncoproteins as described herein in the present invention, wherein the combinatorial oncoproteins are selected from SMO, HFU, ULK3, RAS and ERK12 as potential drug targets in the hedgehog pathway to control or treat pancreatic cancer.

Yet another embodiment of the present invention provides for an in silico method to identify novel combinatorial oncoproteins as potential drug targets that inhibit hedgehog pathway activity in various cancer cell lines required to control or treat cancer in a subject comprising;
(i) reconstructing a novel hedgehog pathway by collating proteins from the various databases; and
(ii) simulating the logical models of the Hedgehog pathway for normal scenario as well as for cancer scenario in Cell Net Analyzer to identify the proteins as drug targets involved in the abnormal activation of hedgehog pathway in the development of cancer.

Yet another embodiment of the present invention provides an in silico method as described in the present wherein the logical analysis of step (ii) comprises;
(i) comparing computationally the number of upstream activator proteins DHH, SHH, IHH, PTCH1, SMO, HIP1, STK36, GLI1, GLI2, GLI3_R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7A, 8A and 9A; number of downstream activated proteins of DISPATCHED, HHAT, BPM_RUNX3, SHH, DHH, IHH, PTCH1, PTCH2, SMO, HIP1, CDO, BOC, GAS1, ULK3, RAS, STK36, HFU, SUFU, TWIST, PKA_A, BTRCP, CKI_A, GSK3, NUC_GLI1, NUC_GLI2, CYCLIN_B, FOXM1, CYCLIN_D, CYCLIN_E, SNAI1, JAGGED2BCL2, CYCLIN_D2 selected from FIGS. 7C, 8C, and 9C; number of upstream inhibitor proteins of DHH, SHH, IHH, PTCH1, SMO, HIP1, STK36, GLI1, GLI2, GLI3_R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7B, 8B and 9B; and number of downstream inhibited proteins of DISPATCHED, HHAT, BPM_RUNX3, SHH, DHH, IHH, PTCH1, PTCH2, SMO, HIP1, CDO, BOC, GAS1, ULK3, RAS, STK36, HFU, SUFU, TWIST, PKA_A, BTRCP, CKI_A, GSK3, NOTCH1, GLI3_R, SKI, NCOR, HDAC, SNO, SIN3A, NUMB, ITCH, NUC_SUFU selected from FIGS. 7D, 8D and 9D of the cancer scenario with each protein of the normal scenario;
(ii) identifying the proteins with significant variations in cancer scenario with respect to the normal scenario; and
(iii) selecting combinations of target proteins from step (ii) for various cancer scenario and perturbing said combination of proteins in the treatment scenario and thereby inhibiting the expression of the output oncoproteins of the hedgehog pathway causing cancer.

Yet another embodiment of the present invention provides an in silico method as described in the present, wherein the number of upstream activator proteins in the cancer scenario is greater than that of the normal scenario thereby effecting the expression of the output oncoproteins.

Yet another embodiment of the present invention provides an in silico method as described in the present, wherein each target protein is assigned ‘0’ or ‘OFF’ and ‘1’ or ‘ON’ to up regulate or down regulate the expression of said protein.

Yet another embodiment of the present invention provides an in silico method as described in the present, wherein the output oncoproteins comprises JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA.

Yet another embodiment of the present invention provides an in silico method as described in the present, wherein the down regulation of output oncoproteins alters the phenotypic outcomes or cellular responses such as Cell proliferation, Cell cycle progression and Endothelial to Mesenchymal transition and also down regulates the pathways such as WNT, NOTCH and Anti-Apoptosis.

Yet another embodiment of the present invention provides an in silico method as described in the present, wherein the combinatorial oncoproteins as potential drug targets comprises (a) combination of SMO, HFU, ULK3 and RAS; and (b) combination of SMO, HFU, ULK3, RAS and ERK12.

Yet another embodiment of the present invention provides an in silico method as described in the present, wherein the type of cancer treated is selected from pancreatic or colon cancer.

Yet another embodiment of the present invention provides an in silico method as described in the present, wherein the databases is selected from KEGG, PATHWAY CENTRAL, BIOCARTA, PROTEIN LOUNGE, NETPATH, GENEGO and other relevant databases.

Yet another embodiment of the present invention provides an in silico method as described in the present, wherein the hedgehog pathway comprises 57 proteins of which 52 are core proteins, 5 cross talk protein molecules obtained from other pathways, 6 cellular or phenotypic expressions and 96 hyper-interactions.

Another embodiment of the present invention provides for an in silico method for selecting cancer treatment regime for colon cancer comprising perturbation of logical states of combination proteins selected from SMO, HFU, ULK3 and RAS from 1 (“ON”) to 0 (“OFF”) of the hedgehog pathway in the treatment scenario to down regulate the expression of GLI transcription factors and subsequently suppressing the expression of output onco proteins such as JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA as well as the phenotypic expressions of the colon cancer cell line.

Another embodiment of the present invention an in silico method for selecting cancer treatment regime for pancreatic cancer comprising perturbation of the combination proteins selected from SMO, HFU, ULK3, RAS and ERK12 from 1 (“ON”) to 0 (“OFF”) of the hedgehog pathway in the treatment scenario to down regulate the expression of GLI transcription factors and subsequently suppressing the output proteins such as JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA as well as the phenotypic expressions of the pancreatic cancer cell line.

Another embodiment of the present invention provides for a use of novel combinatorial oncoproteins comprising SMO, HFU, ULK3, RAS and ERK12 as potential drug targets in the Hedgehog pathway to control or treat cancer.

Yet another embodiment of the present invention provides for use of combinatorial oncoproteins as described in the present invention wherein the cancer is colon or pancreatic cancer.

Yet another embodiment of the present invention provides for use of combinatorial oncoproteins as described in the present invention wherein the combinatorial oncoproteins are selected from SMO, HFU, ULK3, RAS as potential drug targets in the Hedgehog pathway to control or treat colon cancer.

Yet another embodiment of the present invention provides for use of combinatorial oncoproteins as described in the present invention wherein the combinatorial oncoproteins are selected from SMO, HFU, ULK3, RAS and ERK12 as potential drug targets in the hedgehog pathway to control or treat pancreatic cancer.

Another embodiment of the present invention provides for novel combinatorial oncoproteins biomarkers, wherein the biomarkers enable identification of target oncoproteins of the hedgehog pathway for control or treat colon.

Another embodiment of the present invention provides for novel combinatorial oncoproteins biomarkers as described in the present invention wherein the cancer is colon or pancreatic cancer.

Another embodiment of the present invention provides for novel combinatorial oncoproteins biomarkers as described in the present invention wherein the combinatorial oncoproteins biomarkers are selected from SMO, HFU, ULK3, RAS as potential drug targets in the Hedgehog pathway to control or treat colon cancer.

Another embodiment of the present invention provides for novel combinatorial oncoproteins biomarkers as described in the present invention wherein the combinatorial oncoproteins biomarkers are selected from SMO, HFU, ULK3, RAS and ERK12 as potential drug targets in the hedgehog pathway to control or treat pancreatic cancer.

Another embodiment of the present invention provides for an in silico method to identify novel combinatorial oncoproteins biomarkers as potential drug targets that inhibit hedgehog pathway activity in various cancer cell lines required to control or treat cancer in a subject comprising;
(i) reconstructing a novel hedgehog pathway by collating proteins from the various databases; and
(ii) simulating the logical models of the Hedgehog pathway for normal scenario as well as for cancer scenario in Cell Net Analyzer to identify the proteins as drug targets involved in the abnormal activation of hedgehog pathway in the development of cancer.

Yet another embodiment of the present invention provides for an in silico method to identify novel combinatorial oncoproteins biomarkers as potential drug targets as described in the present invention, wherein the logical analysis of step (ii) comprises; (i) comparing computationally the number of upstream activator proteins DHH, SHH, IHH, PTCH1, SMO, HIP1, STK36, GLI1, GLI2, GLI3_R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7A, 8A and 9A; number of downstream activated proteins of DISPATCHED, HHAT, BPM_RUNX3, SHH, DHH, IHH, PTCH1, PTCH2, SMO, HIP1, CDO, BOC, GAS1, ULK3, RAS, STK36, HFU, SUFU, TWIST, PKA_A, BTRCP, CKI_A, GSK3, NUC_GLI1, NUC_GLI2, CYCLIN_B, FOXM1, CYCLIN_D, CYCLIN_E, SNAI1, JAGGED2BCL2, CYCLIN_D2 selected from FIGS. 7C, 8C, and 9C; number of upstream inhibitor proteins of DHH, SHH, IHH, PTCH1, SMO, HIP1, STK36, GLI1, GLI2, GLI3_R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7B, 8B and 9B; and number of downstream inhibited proteins of DISPATCHED, HHAT, BPM_RUNX3, SHH, DHH, IHH, PTCH1, PTCH2, SMO, HIP1, CDO, BOC, GAS1, ULK3, RAS, STK36, HFU, SUFU, TWIST, PKA_A, BTRCP, CKI_A, GSK3, NOTCH1, GLI3_R, SKI, NCOR, HDAC, SNO, SIN3A, NUMB, ITCH, NUC_SUFU selected from FIGS. 7D, 8D and 9D of the cancer scenario with each protein of the normal scenario;
(ii) identifying the proteins with significant variations in cancer scenario with respect to the normal scenario; and
(iii) selecting combinations of target proteins from step (ii) for various cancer scenario and perturbing said combination of proteins in the treatment scenario and thereby inhibiting the expression of the output oncoproteins of the hedgehog pathway causing cancer.

Yet another embodiment of the present invention provides for an in silico method to identify novel combinatorial oncoproteins biomarkers as potential drug targets as described in the present invention, wherein the number of upstream activator proteins in the cancer scenario is greater than that of the normal scenario thereby effecting the expression of the output oncoproteins.

Yet another embodiment of the present invention provides for an in silico method to identify novel combinatorial oncoproteins biomarkers as potential drug targets as described in the present invention, wherein each target protein is assigned ‘0’ or ‘OFF’ and ‘1’ or ‘ON’ to up regulate or down regulate the expression of said protein.

Yet another embodiment of the present invention provides for an in silico method to identify novel combinatorial oncoproteins biomarkers as potential drug targets as described in the present invention, wherein the output oncoproteins comprises JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA.

Yet another embodiment of the present invention provides for an in silico method to identify novel combinatorial oncoproteins biomarkers as potential drug targets as described in the present invention, wherein the down regulation of output oncoproteins alters the phenotypic outcomes or cellular responses such as Cell proliferation, Cell cycle progression and Endothelial to Mesenchymal transition and also down regulates the pathways such as WNT, NOTCH and Anti-Apoptosis.

Yet another embodiment of the present invention provides for an in silico method to identify novel combinatorial oncoproteins biomarkers as potential drug targets as described in the present invention, wherein the combinatorial oncoproteins biomarkers as potential drug targets comprises (a) combination of SMO, HFU, ULK3 and RAS; and (b) combination of SMO, HFU, ULK3, RAS and ERK12.

Yet another embodiment of the present invention provides for an in silico method to identify novel combinatorial oncoproteins biomarkers as potential drug targets as described in the present invention, wherein the type of cancer treated is selected from pancreatic or colon cancer.

Yet another embodiment of the present invention provides for an in silico method to identify novel combinatorial oncoproteins biomarkers as potential drug targets as described in the present invention, wherein the databases is selected from KEGG, PATHWAY CENTRAL, BIOCARTA, PROTEIN LOUNGE, NETPATH, GENEGO and other relevant databases.

Yet another embodiment of the present invention provides for an in silico method to identify novel combinatorial oncoproteins biomarkers as potential drug targets as described in the present invention, wherein the hedgehog pathway comprises 57 proteins of which 52 are core proteins, 5 cross talk protein molecules obtained from other pathways, 6 cellular or phenotypic expressions and 96 hyper-interactions.

Advantageously, the in silico method of the present invention helps to cure the cancers caused by intracellular proteins apart from the sole mutation in GLI or PATCH1 or SMO by providing novel combination of proteins responsible in causing glioma, colon and pancreatic cancer which the prior art methods have failed. Further, the present invention provides an extensive, robust hedgehog pathway that gives an insight about the signalling proteins in the pathway, the interactions and roles of proteins, along with identification of alternate drug targets for glioma, colon and pancreatic cancer. The comprehensive hedgehog pathway of the instant invention can be used for both structural and logical analysis to identify the important proteins that can be used as potential drug targets for glioma, colon and pancreatic cancer.

The newly reconstructed hedgehog signalling pathway for identification of new combinatorial drug targets or pathway signatures provide a more sensible strategy for finding therapeutic targets for cancer that can be useful to the drug industry and experimental biologists to explore the present pathway further.

The present invention may be embodied in other specific forms without departing from the essential attributes thereof, and it is therefore desired that the present embodiments and examples be considered in all respects as illustrative and not restrictive.

Another embodiment of the present invention provides for biomarkers as described herein in the present invention, wherein the biomarkers enable identification of target oncoproteins of the hedgehog pathway for colon cancer.

Another embodiment of the present invention provides for biomarkers as described herein in the present invention, wherein the biomarkers enable identification of the target oncoproteins of the hedgehog pathway for pancreatic cancer.

Another embodiment of the present invention provides for biomarkers as described herein in the present invention, wherein the target oncoproteins for colon cancer comprises the combination selected from SMO, HFU, ULK3 and RAS.

Another embodiment of the present invention provides for biomarkers as described herein in the present invention, wherein the target oncoproteins for pancreatic cancer comprises the combination selected from SMO, HFU, ULK3, RAS and ERK12.

Another embodiment of the present invention provides for use of hedgehog pathway biomarkers as described herein in the present invention for identification of target oncoproteins.

Advantageously, the in silico method of the present invention helps to cure the cancers caused by intracellular proteins apart from the sole mutation in GLI or PATCH1 or SMO by providing novel combination of proteins responsible in causing glioma, colon and pancreatic cancer which the prior art methods have failed. Further, the present invention provides an extensive, robust hedgehog pathway that gives an insight about the signalling proteins in the pathway, the interactions and roles of proteins, along with identification of alternate drug targets for glioma, colon and pancreatic cancer. The comprehensive hedgehog pathway of the instant invention can be used for both structural and logical analysis to identify the important proteins that can be used as potential drug targets for glioma, colon and pancreatic cancer.

The newly reconstructed hedgehog signalling pathway for identification of new combinatorial drug targets or pathway signatures provide a more sensible strategy for finding therapeutic targets for cancer that can be useful to the drug industry and experimental biologists to explore the present pathway further.

The present invention may be embodied in other specific forms without departing from the essential attributes thereof, and it is therefore desired that the present embodiments and examples be considered in all respects as illustrative and not restrictive.

The following description is of exemplary embodiments only and is not intended to limit the scope, applicability or configuration to the invention in any way, Rather, the following description provides a convenient illustration for implementing exemplary embodiments of the invention various changes to the described embodiments may be made in the functions and arrangement of the elements described without departing from the scope of the invention.

### EXAMPLES

### Example 1

### Experimental Methodology

1. Construction of Hedgehog Signaling Networks:

A comprehensive Hedgehog signalling map (FIG. 4 and FIG. 5) was constructed by collating the data based on human cell line and not any particular cell type or disease specific scenario from various biological databases provided [in Table 3].

The cross talks and phenotypic expressions of the HH pathway (hedgehog pathway) named as “Cellular Responses” were connected with output/produced proteins by dotted black arrow. The following are the descriptions of the proteins of each region in the reconstructed Hedgehog signalling network.
1. Extracellular and Membrane: In this region, the three hedgehog ligands viz. Sonic Hedgehog (SHH), Indian Hedgehog (IHH) and Desert Hedgehog (DHH) were bound to the receptor proteins Patched1 (PTCH1) and Patched2 (PTCH2) of a hedgehog target or responsive cell. It was established that in the absence of any of these hedgehog ligands, PTCH1/PTCH2 inhibits trans-membrane G-coupled protein “Smoothened (SMO)” within the cell membrane and the inhibition is withdrawn after the HH ligands bind to the Patched receptors. As a result of this ligand-receptor interaction, SMO is activated and subsequently activates the Serine/Threonine kinase 36 (STK36) which is a major potential activator of Glioma-associated protein (GLI) in cytoplasm in the downstream cytoplasmic region of cell. There are total 3 ligands, 6 extracellular proteins and 4 membrane proteins in Extracellular and Membrane region.
2. Cytoplasmic Proteins: In this region, total 16 protein molecules were included. All the three isoforms of GLI transcription factors GLI1, GLI2 and GLI3 are included. GLI were found in Cytoplasm as well as in Nucleus and was the main target component protein for Hedgehog pathway activation. Other proteins in this region that directly or indirectly influence the three isoforms of GLI protein in the cytoplasm were also identified. These proteins are Human Fused (HFU), Unc-51-like kinase 3(ULK3), ERK1/2, RAS and TWIST. The non-core HH pathway proteins ERK12, RAS, TWIST, FAS and NOTCH1a having significant direct interactions with core proteins GLI1, GLI2 and SMO were also further identified. Mutation or over expression of these proteins activated GLI in cytoplasm without the help of any Hedgehog ligands. Protein Kinase A (PKA), Beta-transducin repeat-containing protein (BTRCP), Casein kinase isoform alpha (CKIα), Glycogen synthase kinase-3 (GSK3) which are GLI repressors were further included in the present network.
3. Nuclear Proteins: In the nuclear region of the Hedgehog pathway map, 13 molecules were included mainly transcription factor, co-activator or co-repressor. The activated transcription factors GLI1, GLI2 and GLI3 translocate into the nucleus as Nuclear GLI1 (NUC_GLI1), Nuclear GLI2 (NUC_GLI2) and GLI3 active (GLI3_A) respectively and helped to transcribe various hedgehog target genes with the help of transcription co-activators Nuclear STK36 (NUC_STK36) and Dual specificity tyrosine-phosphorylation-regulated kinase 1 (DYRK1) proteins. Further, few transcription co-repressors in the nucleus that down regulate the GLI transcription factors comprising the proteins such as Nuclear SUFU (NUC_SUFU), NUMB, ITCH, SKI, Nuclear Receptor Co-repressor (NCOR), SNO, HDAC and SIN3A, were included in the network. In nucleus NUC_GLI1 transcription factor transcribes the genes ptch1, hip1, gli1 along with several other responsive genes of this pathway. In order to reduce the complexity in the pathway figure, however no gene or m-RNA was included in this nuclear region.
4. Output Proteins: This region does not specify any cellular location and was included separately to identify the proteins produced at the end of Hedgehog pathway. The present signalling network is an input-output system where the ligands and extracellular proteins are the inputs, the proteins produced as a response to these inputs at the end of this pathway are considered as Output proteins. There are total 15 proteins including GLI1, PTCH1 and HHIP as Output proteins. The total numbers of output proteins were highest compared to any hitherto published human specific Hedgehog pathway map. All proteins except PTCH1, HHIP and GLI1 since after translation, these three proteins translocate to their corresponding cellular locations and perform their activity in the pathway. In order to show this feedback mechanism, their color was kept similar to the color coded in their actual cellular location. Production of PTCH1 and HHIP proteins in this pathway switch “ON” a “negative feedback” mechanism and thus control further hedgehog pathway activation through ligand dependent way.
5. Cellular Responses: In order to show the cross connections of the output proteins with the other pathway or cellular functions, this section was kept at the end of the pathway figure. There are 6 cellular responses included which are Cell Proliferation, Cell cycle progression, Anti-Apoptosis, Epithelial-Mesenchymal transition (EMT), Wnt signal and Notch signal. The connections of produced proteins with these cellular responses are shown by black dotted arrow in the pathway figure.

2. Structural Analysis

To find out the structure and topological features of the instant Hedgehog signalling network, ‘Graph theory’ was used for analysis. In the present study, the whole signalling pathway was considered as a network where the signal from the Hedgehog ligands traverses from extracellular region to the nucleus of a target cell via various cytoplasmic intermediate proteins. In order to show only the connections of the proteins within the Hedgehog map, the Cellular responses were not included in the graph theoretical model. The whole network picture is shown in FIG. 5. The coloured circles represent the nodes or proteins of the pathway and the black arrows indicate an edge or connection between two nodes of the network. The nodes are coloured according to their sub cellular locations in the cell (FIG. 4).

The output proteins GLI1, PTCH1 and HHIP are not shown in the “output” region but are presented as reverse connections from NUC_GLI 1 to the GLI 1 of cytoplasm and to PTCH1 and HHIP of membrane region. The size of the nodes in this network (FIG. 5) was assigned according to their total number of connections or degree value.

Since, GLI1 in cytoplasm has the highest number of total degree in the network; therefore the size of this node in the network was largest among all the other nodes. FIG. 5 further indicates that the Hedgehog signals from the extracellular and membrane proteins converged to the particular cytoplasmic proteins (i.e GLI1 and GLI2) to activate it and after its activation these proteins sent the signals (actually translocate into the nucleus) to activate the production of the various target genes/proteins (like OPN, BCL2, GLI1, HHIP etc.) at the downstream of hedgehog pathway. The flow of hedgehog signal from extracellular-membrane region to the downstream target proteins of hedgehog pathway mainly depends on the intermediate cytoplasmic GLI proteins. Therefore, the canonical hedgehog pathway is also called as ‘GLI mediated hedgehog pathway’.

Further analysis of the network was carried out from three perspectives: i) Connectivity ii) Centrality and iii) All pairs shortest path.

Connectivity analysis: The analysis was performed to know the number of connections of each protein with all other proteins in the network. Three types of parameters (IN DEGREE, OUT DEGREE and TOTAL DEGREE) were used where these three parameters for each protein of the Hedgehog signaling network was disclosed in FIG. 6A. The heat map representing the values of the parameters (IN-DEGREE, OUT-DEGREE and TOTAL DEGREE), shows the protein row wise according to their cellular locations (top to bottom) and the parameter values column wise. The average IN and OUT DEGREE of the network was calculated as 2.45 and the average total degree was 4.91. In order to identify the important proteins from this heat plot on the basis of the connectivity parameters, the proteins were extracted which had parameter values higher than their corresponding average values. All the extracted significant proteins on the basis of this hypothesis are listed in Table 7 below. It was found that there are total 19, 10 and 23 proteins which have higher values than the average IN-DEGREE, OUT-DEGREE and TOTAL-DEGREE, respectively.

Table 7 show that receptor protein PTCH 1 and two transcription factors GLI 1 & GLI2 have higher IN-DEGREE values compared to the other proteins in the entire network, which may be due to their high regulation or interaction with other upstream proteins in the hedgehog signaling network. PTCH1 showed higher IN-DEGREE because most of the extracellular signals passed through this receptor protein to trigger the activation of SMO protein in membrane. On the other hand the cytoplasmic GLI1 and GLI2 had high IN-DEGREE value as these proteins were the most important proteins in the network to activate the pathway.

Also, among the three hedgehog ligands, Sonic hedgehog (SHH) had the highest IN-DEGREE value as its interaction with PTCH1 and PTCH2 receptors was highly dependent on the proteins DISPATCHED, HHAT, CDO, BOC and GAS1 at the extracellular region of hedgehog target cell. The proteins in the nucleus like NUC_GLI1, NUC_GLI2, DYRK1 etc. had highest out-degree value compared to the other proteins in the network. Mainly the output proteins were connected to the outgoing connections or edges of these nuclear proteins in the network structure. Due to the presence of the higher number of outgoing connections from the nuclear proteins to the output proteins, the OUT-DEGREE values of these proteins were increased in comparison to the other proteins in the whole network. Except the nuclear) proteins, the proteins from the other sub cellular locations or regions however did not show significant OUT-DEGREE values.

Further, the proteins were extracted which had the TOTAL-DEGREE higher than the average total-degree 4.91. Table 7 shows that in extracellular and ligands region PTCH1, HHIP, SHH, IHH have significant number of connections or total degree than the average total degree in the network indicating the effective role of these proteins in the transmission of the hedgehog signal from extracellular to the intracellular region of a cell. Further, it is evident from FIG. 6A, that GLI1 has the highest TOTAL-DEGREE value among all other proteins in the Hedgehog signaling network wherein out of 57 proteins in the network, it is connected to 30 proteins. Therefore in terms of signaling network, it was considered as the biggest ‘Hub’ in the entire network. Similarly, in nuclear region NUC_GLI1, NUC_SUFU and NUC_STK36 formed the other larger hubs in the nucleus thus controlling the production of various target proteins of Hedgehog pathway.

Centrality measurements: “Centrality Values (Eigenvector, Closeness and Betweenness)”, are the most useful parameters, used to determine the relative importance of a node within a network.

To identify the proteins according to their importance in the newly constructed hedgehog signalling network (in FIG. 6B i.e. first column of the heat plot matrix), the present inventors relied upon ‘Eigenvector centrality’. The principle behind this parameter is that a node is considered as an important node if it is connected to the other important nodes in the network. It was observed that GLI1 and PTCH1 had high Eigenvector centrality score, but GLI2 had very poor score though it has large number of connections or degree in the network., GLI2 was observed to be connected to NUC_GLI2, FAS, HFU, SUFU, PKA_A, BTRCP etc. On the other hand, GLI1 was connected with another important node or protein NUC_GLI1 in the network which regulates the expression of most of the output proteins in the network and comparably has higher number of connections than NUC_GLI2. this signifies that GLI1 is connected to another most important protein NUC_GLI1 in the network. in addition, proteins such as SMO, GLI3_Repressor (GLI3_R), GLI3_Active (GLI3_A) were observed as other t important nodes after GLI1 and PTCH1, though they have lower number of connections or neighbors within the network.

The inventors contemplated that GLI1 has the highest Betweenness and Closeness centrality score among all the other proteins in the Hedgehog signalling network (FIG. 6B) because of large numbers of shortest paths passing between two nodes which are connected to all other proteins with the minimum number of connections in the network. Besides GLI1, other proteins like NUC_GLI1, SMO, STK36 and PTCH1 were observed to have high Betweenness centrality score. On the other hand proteins such as NUC_SUFU, NUC_STK36, DYRK1, NUMB and ITCH showed high Closeness centrality score after GLI 1.

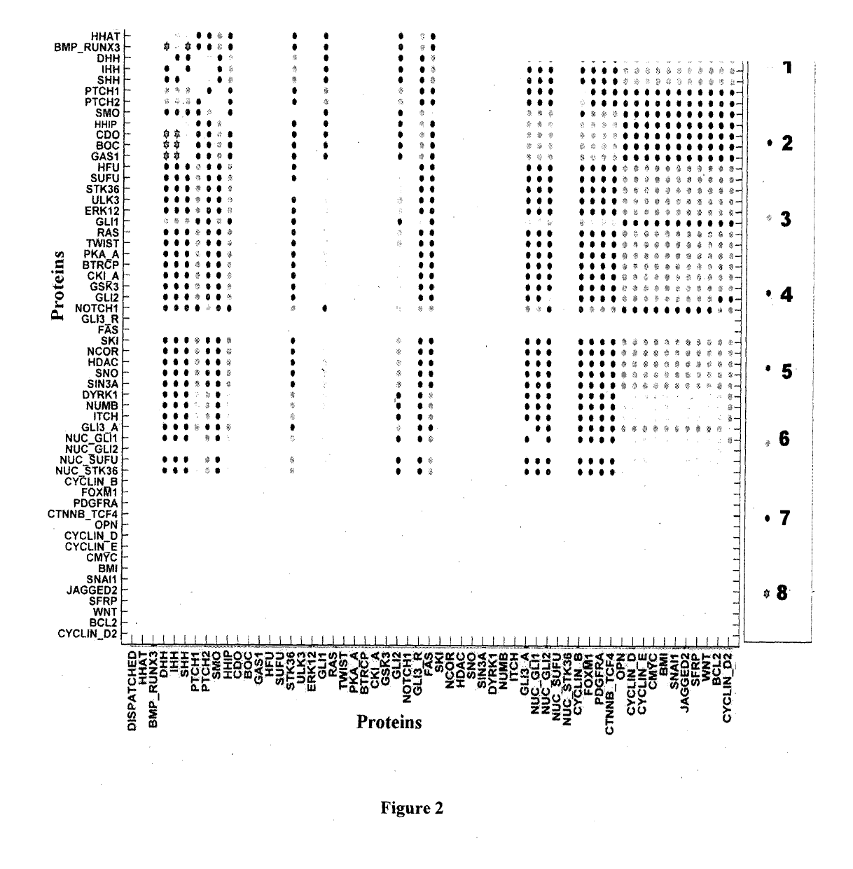

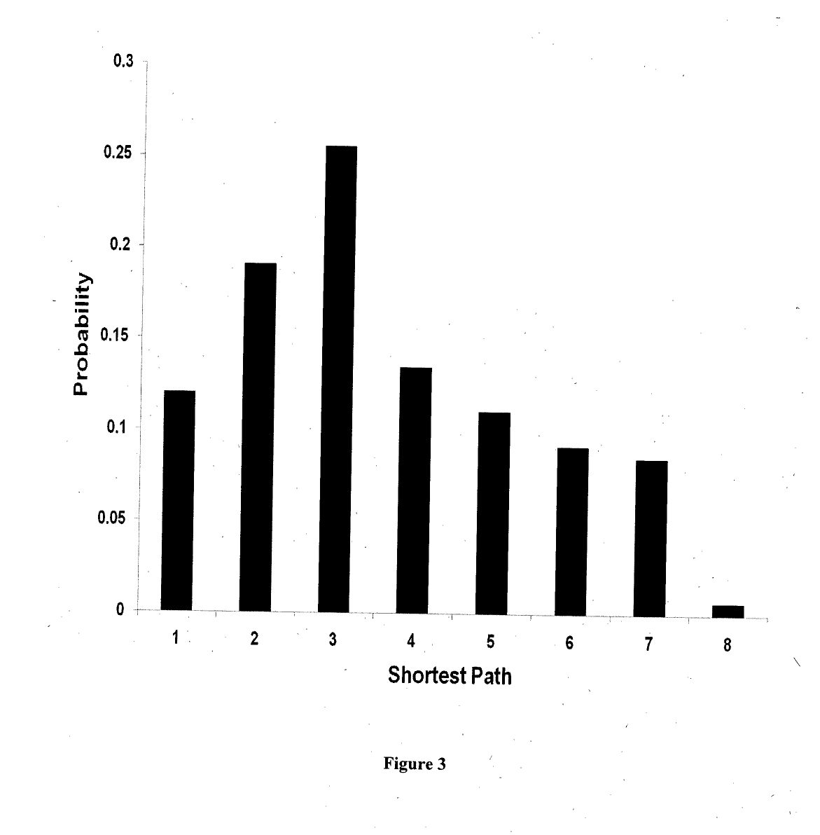

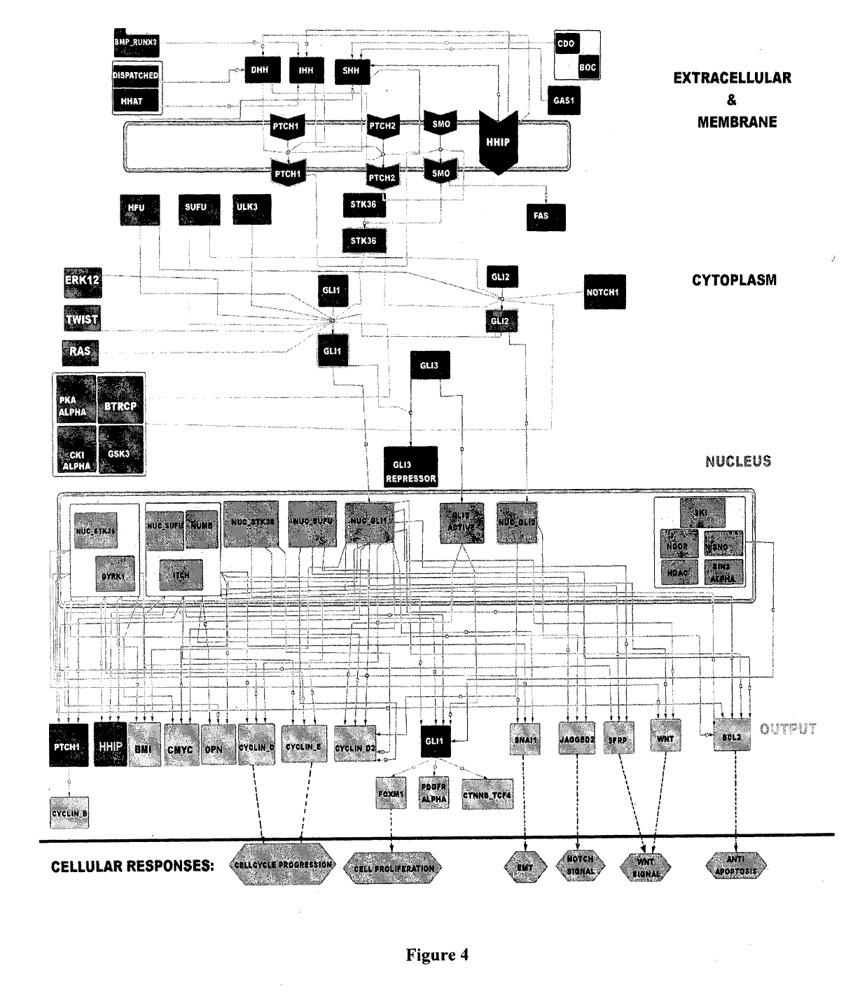

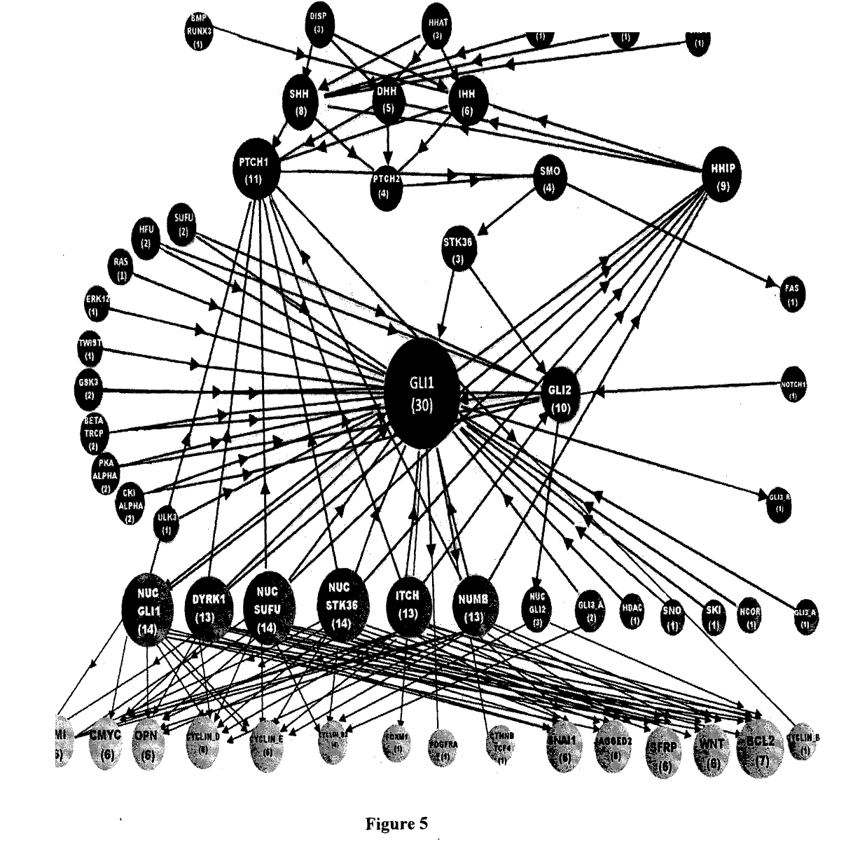

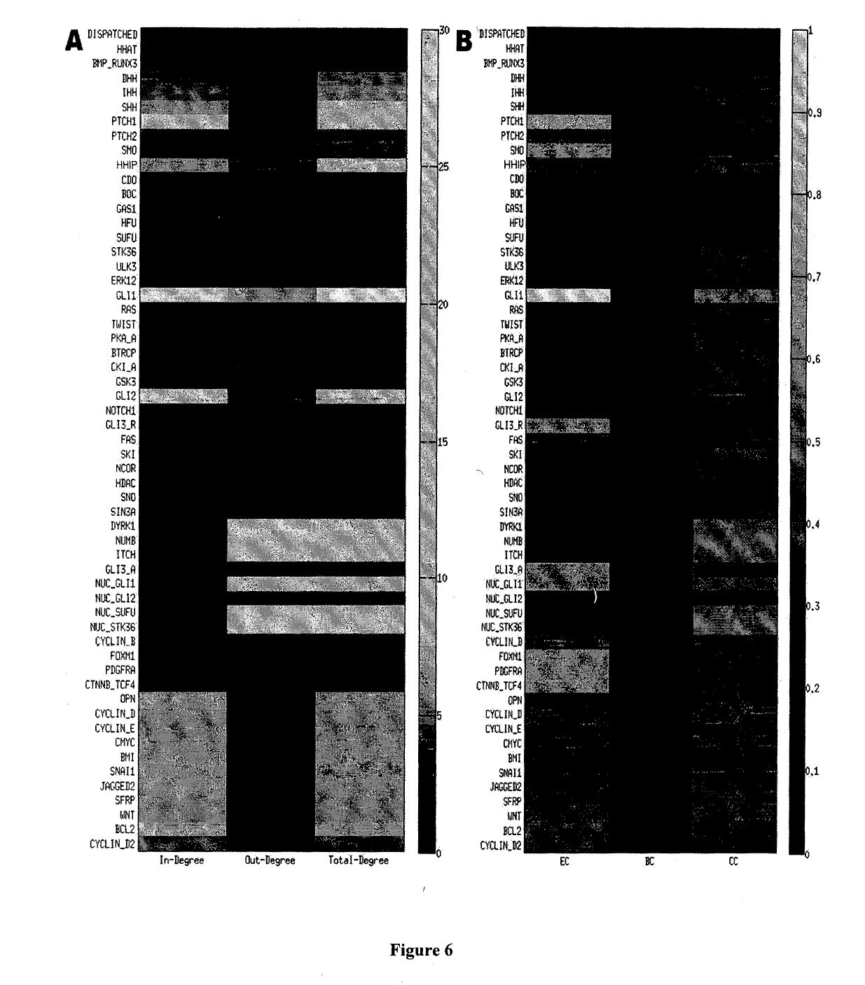

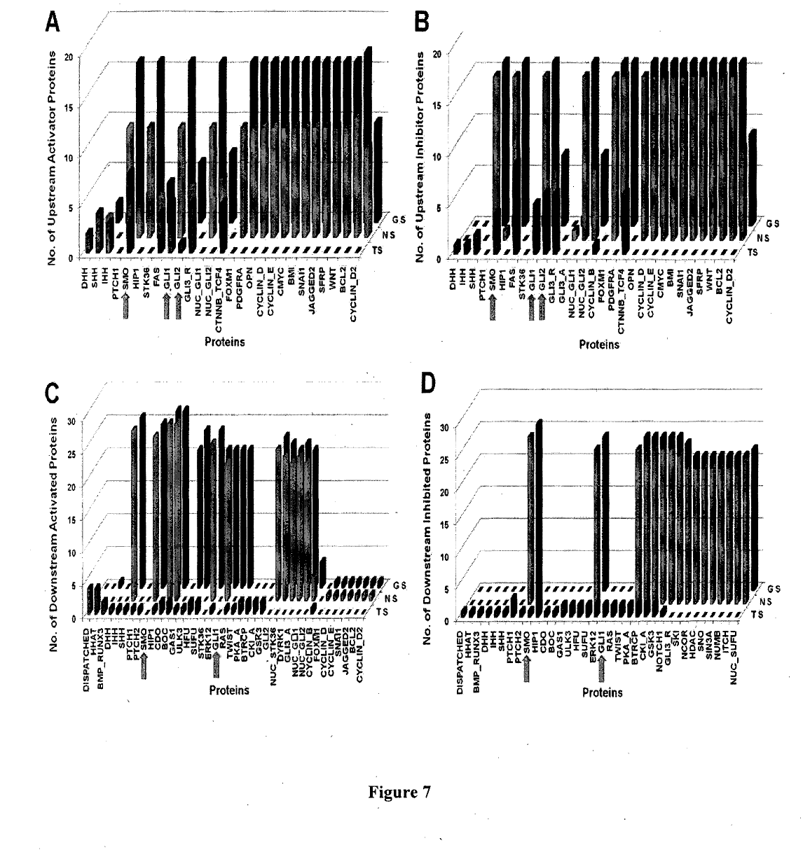

Linear shortest paths: This parameter was used to calculate the shortest paths between every pair of nodes or proteins in the hedgehog pathway. The inventors extracted the frequency of the shortest paths of the present Hedgehog signalling network and presented the distribution in FIG. 3. The frequency of the shortest path ‘3 (Three)’ in the network was observed highest which signified that most of the proteins in the hedgehog signalling network are connected with each other on an average by 3 intermediate steps. From FIG. 2 it was inferred that in order to transmit the signal after the bindings of PTCH1/2 proteins with ligands (SHH, DHH, IHH) to GLI1 or GLI2 proteins in the cytoplasm, it took only 3 (three) intermediate steps or links (i.e. PTCH1/PTCH2→SMO→STK36→GLI1/GLI2). Similarly, in order to initiate the production of the output proteins of the hedgehog pathway in the nucleus by the transcription factors GLI1, GLI2 or GLI3_ACTIVE, it required only 2 intermediate steps or links i.e. GLI1/GLI2→NUC_GI1/NUC_GLI2→CYCLIN_D/CMYC/BCL2. it was further observed that the proteins identified as ‘important hubs’ such as GLI1, PTCH1, NUC_GLI1 etc. were are also connected by shorter number of links to each other in the network. Therefore, it was inferred that most of the important or highly connected proteins in hedgehog signalling network were well connected with each other by lower number of connections or links, which also signifies the robustness of the present network.

### Logical Analysis:

The inventors simulated the logical models of the Hedgehog pathway for normal scenario as well as for three different types of cancer i.e. Glioma, Colon and Pancreatic cancer in CellNetAnalyzer. to identify the proteins involved in the abnormal activation of hedgehog pathway in the development of these three types of cancers.

Accordingly, the entire Hedgehog signaling network was organized into a three layered system of input, intermediate and output, with input signals orchestrating cellular responses to output via intermediate molecules. To visualize and analyze the Hedgehog signal transduction network, the inventors constructed the Logical or Boolean Interaction Hyper-graph with large number of nodes and interactions or hyper-arc. In the Boolean network each node represented a protein (Ligands, Receptors, kinase or Transcription factor) or cellular response (Cell Proliferation, Cell cycle progression, Wnt Pathway etc.) whose state could be either be ‘0’ (OFF) or ‘1’ (ON). Depending on the cellular function and/or location, the proteins were classified as active (ON) or inactive (OFF). The entire simulation of Boolean modelling was performed in Cell Net Analyzer and the following steps were followed during the logical simulation.
1. Selection of the states of input and output proteins
2. Construction of Boolean or logical equations
3. Simulation and perturbation using different logical states.

### Perturbation Analysis and Model Validation

As shown in FIGS. 7-9, the normal Hedgehog scenario were presented along with three types of cancers Glioma, Colon and Pancreatic cancer as well as their perturbed or combinatorial drug treated scenarios respectively. In each figure, the proteins were represented along X-axis and three types of scenarios i.e. Normal Scenario (NS); Cancer Scenario [Glioma (GS); Colon (CC); Pancreatic (PC)]; Perturbed or drug treated Scenario (TS) were along the Y-axis and the number of upstream activator/inhibitor proteins (A/B) or downstream activated/inhibited (C/D) proteins were along the Z-axis.

In case of ‘Canonical hedgehog pathway’ (i.e. NS or Normal Scenario), it was found that at time scale 2 Sonic hedgehog (SHH) was activating overall 26 different proteins (from FIGS. 7C, 8C and 9C) in the pathway directly or indirectly after being activated by the upstream proteins DISPATCHED, HHAT, CDO, BOC etc. Besides, it was found that SMO, STK36 were activating overall 25 and 24 other protein molecules in the pathway respectively. Similarly it was observed that in the normal scenario, cytoplasmic proteins such as GLI1 and GLI2 were activating around 21 and 23 proteins respectively. FIG. 7B-9B showed that the upstream inhibitors (nearly 20 protein molecules) such as PTCH2, SUFU, BTRCP, GSK3, PKA, CKI_A, NCOR, HDAC, SNO, SIN3A, NUMB and ITCH inhibited the activation or production of GLI1 in cytoplasm. This inhibition helped to control the over expression of GLI1 in a normal cell. The interactions between the activators and inhibitors in a canonical hedgehog pathway helped to control their abnormal activation, and consequently regulated the over production of various downstream proteins, which further resulted a cancerous situation in a normal and healthy cell.

### Example 2

### Comparison Between Normal, Cancer and Perturbed Scenarios for Glioma, Colon and Pancreatic Cancers

### TS: Treated Scenario; NS: Normal Scenario; GS: Glioma Scenario

Glioma Scenario: The comparison between Normal, Cancer and Perturbed scenarios for Glioma is provided in Table 4 and in FIGS. 7A-7D.

Form the analysis of the data in Table 4 and FIGs. 7A to 7D, the present inventors identified the key proteins that can be used as target proteins for the perturbation analysis or combinatorial drug treatment scenario (TS).

Combining data, indicates that proteins such as SHH, SMO, STK36, RAS, TWIST, ERK12, HFU, ULK3 activated the GLI transcription factors in the cytoplasm of Glioma cell and due to this activation, the output proteins were over-expressed at the end of the present pathway.

FIG. 7A depicts that in gliaoma scenario, the number of upstream activator proteins of DHH, SHH, IHH, PTCH1, SMO, HIP1, STK36, GLI1, GLI2, GLI3_R, NUC_GLI1, NUC_GLI2, CTNNB_TCF4 etc. were higher than that of the normal scenario and in case of perturbation the number of upstream activator proteins was zero. The total numbers of upstream inhibitor proteins remain unchanged while comparing the Normal and Glioma scenarios (FIG. 7B), in FIGS. 7C and 7D, potential activator and inhibitor of the HH pathway activated and inhibited less number of proteins in the down steam region of the pathway respectively in the perturbed scenario.

Colon Cancer: The comparison between Normal, Cancer and Perturbed scenarios for Glioma is provided in Table 4 and in FIGS. 8A-8D.

Taken together, the data indicates that in case of Colon cancer, along with IHH and SHH ligands, activated RAS/RAK pathway also up-regulated the activity of the GLI proteins in colorectal cancer cell. As a result the activation level of GLI1, GLI2 and GLI3_A proteins was greater than the normal scenario (FIG. 8A to 8C). Also, the total number of upstream activators of the output proteins (in FIGS. 4 and 5) of Hedgehog pathway increased in colon cancer scenario (by comparing the normal or “NS” and colon cancer “CC” scenarios of FIG. 8A).

The inventors further perturbed the activation signal from SHH and IHH via PATCHED (PTCH1/2) and SMO proteins to GLI transcription factors and the interactions between HFU, ULK3 and RAS with GLI proteins. This was done by putting the logical state of SMO, HFU, ULK3 and RAS as ‘0’ (Zero) and performing the combinatorial drug perturbation or Treatment simulation (TS). It was observed that the total number of activated proteins by GLI1, GLI2 and GLI3 decreased and the expression of the HFU, ULK3 and RAS proteins were blocked (comparing Colon cancer and Treated scenario of FIG. 8C).

FIGS. 8A and 8B, show that the activation and inhibition level of GLI transcription factors in nucleus and cytoplasm by the upstream proteins were reduced; as a result, the activation of output proteins were blocked.

Pancreatic Cancer: The expression of IHH, PTCH1 and SMO in pancreatic cancer cell line signify their role in hedgehog pathway activation. Hence, in order to completely repress the over expression of GLI proteins in cytoplasm, the inventors perturbed the logical states of RAS and ERK12 in the present Boolean model of pancreatic cancer scenario. The logical state of HFU (Human homologue of fused protein) and ULK3 were perturbed as these are the common and essential auto-phosphorylated kinase proteins for enhancing the activation of GLI1 and GLI2 in cytoplasm. Perturbation of these proteins caused the suppression of GLI transcription factors in cytoplasm and subsequently inhibited the Hedgehog target or output genes/proteins. The inventors observed that the SMO, HFU, ULK3, RAS, ERK12 proteins were repressed and subsequently the expression of GLI transcription factors in cytoplasm was down-regulated. The down-regulation of GLI proteins in cytoplasm caused the down regulated productions or the transcription of various target onco-genes or onco-proteins of Hedgehog pathway like BMI, FOXM1 etc. The expression of various output proteins like OPN, BMI, SNAI1, JAGGED2, PDGFRA, was not observed in the present drug treated perturbation scenario (TS).

In Pancreatic cancer model, it was observed that GLI2 had higher number of downstream activated proteins compared to GLI1 (FIG. 9C) as it has an activation effect on GLI1, although the Eigenvector centrality of GLI2 was low compared to GLI1. The experimentally observed expression data of each protein for all three types of cancer is presented in FIGS. 10A-10C. The model was simulated in two ways to validate with these experimental observations for three types of cancers. In Simulation 1 (SIM1), the inventors considered the logical states mentioned in (Table 4). In Simulation 2 (SIM2), the expression of input proteins observed from the experimental data (EXP) for each of these cancer types was considered. The experimental data indicated that up regulation of IHH, RUNX3, SMO, STK36, TWIST, ERK12, RAS and down regulation of tumor suppressor onco proteins SUFU were co-occurring in Glioma grade IV cell line (first column of FIG. 10A). Similarly, in Colon cancer cell line, up regulation of SHH, GLI1, PDGFRA were co-occurring (first column of FIG. 10B). On the other hand up regulation of SHH, STK36, ERK12, RAS and down regulation of SUFU were co-occurring in Pancreatic cancer cell line. In the second simulation (Simulation 2), all these co-occurrences were considered as initial states to provide biologically realistic predictions.

### Example 3

### Validation of Glioma, Colon and Pancreatic Scenarios

In case of Glioma Grade IV cell line, the inventors found the expression level (UP as Red and DOWN as Blue) of 33 proteins out of 57 proteins (first column of FIG. 10A) from previously done experimental result on Glioma cell line [24]. Rest of the proteins showed undetermined expression level and were grouped into the lower portion (Light Blue). Within these 33 determined proteins, the simulation (SIM1; second column of FIG. 10A) correctly predicts the expression level of 22 proteins (66.66% accuracy). This result also signify the effect of co-occurrence of the over expression of the activator proteins HFU, RAS, TWIST of hedgehog pathway in Glioma Grade IV cell line. Further using the experimental expression data [24], Simulation 2 (third column of FIG. 7A) was performed and the outcome with both Experiment (EXP) and Simulation 1 (SIM1) (Table 6 were compared. Comparing the results of Simulation 2 (SIM2) with Experimental data (EXP) (first and third column of FIG. 10A), it was found that out of 33 experimentally determined proteins, the current method correctly predicts the expression levels of 25 proteins (75.75% accuracy). On the other hand, while comparing the simulation result between Simulation 1 and Simulation 2 (first and second column of FIG. 10A), it was found that out of 57 proteins, 54 proteins showed same expressions levels thus having accuracy 94.37% (Table 6). Therefore, in both the cases the present model show promising predictions as compared to experimental data of Glioma Grade IV cell line. Hence the combination of drug targetable proteins identified from the drug treatment scenario (TS) of Glioma model could be used as drug targets for the treatment of Glioma Grade IV specific cell line.

In case of Colon cancer scenario, the protein expression of colon cancer cell line data were considered and the up regulation of SHH, PTCH1, HHIP, GLI1, GLI3_Active and PDGFRA were identified from the previously done experimental result on Colon cancer cell line (the first column of FIG. 10B) [25]. The expression levels of rest of the proteins were considered as “Not available” and were grouped separately in the first column of FIG. 7B. Within these 5 determined proteins the current simulation (SIM1)) correctly predicted their expressions with 100% accuracy (the second column of FIG. 10B). Using the present simulation the inventors were able to find out the expressions levels of the other proteins, whose expression levels were not available in the experimental data, which is one of the advantages of the current model simulation. Further, using the experimental expression data, the inventors performed Simulation 2 (the third column of FIG. 10B) and compared the outcome with both Experiment (EXP) and Simulation 1 (SIM1) (Table 6). In both the cases same expression levels of the proteins (100% accuracy) were observed, which strongly validates the current model on Colon cancer scenario. Similarly, the present model simulation for Pancreatic cancer cell line also showed significant high accuracy while validating with the experimental data [26]. The inventors were able to extract the expressions levels of 44 out of 57 proteins of hedgehog model in pancreatic cancer cell line from the published microarray expression data. The rest of the proteins were grouped into separately (light blue) in the first column of FIG. 10C. The up regulation of HFU, ERK12, RAS were observed in the microarray expression data (the first column of FIG. 10C). Within these 44 determined proteins, first simulation (SIM1) correctly predicted the expression levels of 25 proteins with 56.80% accuracy. Comparing the expressions of the proteins in microarray expression data (EXP) and Simulation 2 (SIM2) (the first and third columns of FIG. 10C), it was found that out of 44 determined proteins of micro array expression data, Simulation 2 correctly predicted the expressions level of 32 proteins with 72.72% accuracy (Table 6). On the other hand while comparing the simulation results between Simulation 1 and Simulation 2, the inventors found that out of 57 proteins, the current simulation correctly predicted the expression levels of 47 proteins with 82.45% accuracy (See Table 6). Thus the expressions levels mentioned in Table 4 were sufficient to simulate the pancreatic cancer cell line more close to reality.

These results clearly signify that the expression values mentioned in Table 4 for all the three cancer scenarios were almost correctly considered and validated the present model simulations. Further the target proteins such as RAS, ERK12, HFU, ULK3, identified from the treatment scenario, are also been over expressed in the experimental data.

### Example 4

### Comparison Between SMO Inhibition and Combinatorial Drug Treatment

Further to check the effectiveness of SMO inhibition in the treatment of three types of cancers the present invention provides a model simulation by only inhibiting the SMO expression levels (in-silico treatment of cancer cell with SMO inhibitors) and found that SMO inhibition alone was not able to down regulate the activity of some onco-proteins such as GLI1, GLI2, GLI3_Active and also other target output proteins connected by these proteins through different transcriptional and translation activities.

Therefore to identify the alternative pathways or connections present in the hedgehog pathway, which activates the GLI transcription factors after inhibiting SMO, calculation of the dependency matrices was carried for each cancer scenarios, where only the SMO activations was blocked. Using this information and from the structural analysis (i.e. the analysis of Shortest paths) of hedgehog signalling network, the alternative pathways which were causing the GLI activations in each type of cancer scenarios were identified. These alternative pathways (solid arrows) are shown in FIG. 11D-11F for Glioma, Colon and Pancreatic cancer cell line respectively. Although the activation pathways (broken arrows) from SMO to GLI1 and GLI2 via STK36 protein were blocked by the effect of SMO inhibitor in each cancer scenario, it was observed that HFU could also activate GLI1 and GLI2 in each cancer scenario (solid arrows). Also, in Glioma scenario, the activation pathways (solid arrows in FIG. 11D) were identified from RAS, ERK12, ULK3 and TWIST to GLI1 that could up regulate this protein, whereas the activation pathways (solid arrows in FIG. 11E) from RAS, ULK3 to GLI1 in Colon cancer scenario or the activation pathways (solid arrows in FIG. 11F) from RAS, ULK3 and ERK12 to GLI1 in pancreatic cancer scenario were observed as the alternative pathways to up regulate the GLI1 protein.

Identification of these alternative pathways clearly showed that in order to suppress the hedgehog pathway activity completely in these three types of cancer cell lines, only the SMO inhibition was not effective, as there were other molecules/proteins which were still activating the pathway. The in-silico combinatorial treatment (i.e. Treatment scenarios) of the current invention has accounted this constraint and as a result the present invention provides combinatorial drug target therapy to completely shut down the hedgehog pathway activity. Using this approach, it was possible to down regulate the activity of GLI proteins as well as the up regulation of the output onco-proteins (the treatment scenarios of FIGS. 7, 8 and 9) by perturbing the over expressions of few optimal combinations of proteins (SMO, GLI1, GLI2 in Glioma Grade IV cell line; SMO, HFU, ULK3 and RAS in Colon cancer cell line; and SMO, HFU, ULK3, RAS and ERK12 in pancreatic cancer cell line). In order to compare the SMO inhibition with the proposed combinatorial treatment, simulation results are shown in FIG. 11 as expression matrices for these three cancer types. The second column of FIG. 11A, show the combinatorial inhibition of SMO, GLI1 and GLI2; which establish the down regulation of various onco proteins including GLI transcription factors. The invention also simultaneously disclose the up regulation of few proteins like the repressor form of GLI3 (i.e. GLI3_R), FAS and CTNNB_TCF4 complex, which were also important for inhibiting the uncontrolled cellular proliferations. Similar results were observed in the Treatment scenarios by inhibiting SMO, HFU, ULK3 and RAS in Colon cancer cell line and SMO, HFU, ULK3, RAS and ERK12 in Pancreatic cancer cell lines (the second columns of FIGS. 11B and 11C).

Comparing the cancer scenarios with normal scenario, through novel and expansive constructed Hedgehog signalling pathway and its computational study, the present invention provides a new therapeutic strategy to inhibit the Hedgehog pathway by targeting some novel combination of proteins as future drug targets. Accordingly, the present method successfully identifies minimal combinations of proteins which may be used for further in-vitro and in-vivo analysis as new combinatory drug targets that opens up new avenue to control different cancers especially glioma, colon cancer and pancreatic cancer.

TABLE 1
Name of the Databases used to collate information
for constructing the new Hedgehog Pathway

| Sr. | Name of the | |
|---|---|---|
| No. | Database | Available at |
| 1. | KEGG | http://www.genome.jp/kegg/pathway.html |
| 2. | WikiPathways | http://www.wikipathways.org/index.php/WikiPathways |
| 3. | Millipore | http://www.millipore.com/pathways/pathviewer.do?pathwayId=163 |
| 4. | Invitrogen | http://products.invitrogen.com |
| 5. | Reactome | http://www.reactome.org/ReactomeGWT/entrypoint.html |
| 6. | HPRD | http://www.hprd.org |
| 7. | Netpath | http://www.netpath.org/ |
| 8. | APID | http://bioinfow.dep.usal.es/apid/index.htm |
| 9. | Biocompare | http://www.biocompare.com |
| 10. | GeneGo | http://www.genego.com |
| 11. | Applied Biosystem | http://www5.appliedbiosystems.com/tools/pathway |
| 12. | Pathway Central | http://www.sabiosciences.com/pathway.php?sn=Hedgehog |
| 13. | CNPD | http://cpdb.molgen.mpg.de/ |
| 14. | Cell Signaling | http://www.cellsignal.com/ |
| | Technology | |
| 15. | Pathway Studio | http://www.ariadnegenomics.com/products/pathway-studio |
| 16. | Biocarta | http://www.biocarta.com/genes/index.asp |
| 17. | Biomodels | http://www.ebi.ac.uk/biomodels-main |
| 18. | PID-NCI | http://pid.nci.nih.gov |
| 19. | CancerCellMap | http://cancer.cellmap.org/cellmap/ |
| 20. | EBI- | http://www.ebi.ac.uk/arrayexpress/ |
| | ARRAYEXPRESS | |
| 21. | PathwayCommons | http://www.pathwaycommons.org |

TABLE 2
Comparative statistics of number of species and interactions
for Hedgehog pathway in different database

| | Number | Number of | |
|---|---|---|---|
| | of Species | Species taken | Number of |
| Database | present | in our model | Interactions |
| KEGG | 18 | 18 | 13 |
| Protein Lounge | 12 | 12 | 14 |
| Biocarta | 10 | 10 | 15 |
| Netpath | 31 | 14 | 57 |
| GenGo | 33 | 8 | 43 |
| Pathway Central | 14 | 14 | 14 |

| | | |
|---|---|---|
| Reconstructed | 57 | 96 |
| Hedgehog | | |
| Pathway in this | | |
| study | | |

TABLE 3
Abbreviations and Detail information about the Proteins and Cellular
Responses involved in the newly constructed Hedgehog pathway

| | | |
|---|---|---|
| SHORT NAME USED | | |
| IN MODEL | FULL NAME | DOCUMENTATION |

I. Extracellular and Membrane Proteins

| | | |
|---|---|---|
| BMP_RUNX3 | Bone Morphogenetic | BMP-RUNX3 signaling induces |
| | Protein and Runt related | expression of IHH in surface |
| | transcription Factor 3 | differentiated epithelial cells of |
| | | stomach and intestine. |
| DISPATCHED | Dispatched | Dispatched regulates the release and |
| | | extracellular accumulation of |
| | | cholesterol-modified hedgehog |
| | | proteins and is hence required for |
| | | effective production of the |
| | | Hedgehog signal. |
| HHAT | Hedgehog Acyltransferase | HHAT is a hedgehog modifier |
| | | which induces lipid modification to |
| | | generate mature peptides. Hedgehog |
| | | proteins with lipid modification are |
| | | then released from producing cells |
| | | by Dispatched homologues. |
| DHH | Desert Hedgehog | Three Hedgehog ligands |
| IHH | Indian Hedgehog | (homologues proteins) of Hedgehog |
| SHH | Sonic Hedgehog | pathway considered as Input |
| | | Proteins in this model. |
| PTCH1 | Patched1 | Two homologue of receptor protein |
| PTCH2 | Patched2 | Patched. In the absence of hedgehog |
| | | ligands these proteins inhibit |
| | | another trans membrane protein |
| | | Smoothened (SMO). |
| SMO | Smoothened | G-protein coupled receptor that is |
| | | normally suppressed by Patched |
| | | receptors but is activated in the |
| | | presence of Hedgehog ligands |
| | | (SHH, DHH, IHH). |
| HHIP | Hedgehog Interacting | Regulates the amount of Hedgehog |
| | Protein1 | ligand that can bind to Patched |
| | | receptors |
| CDO | Belong to Immunoglobin | CDO and BOC represent a |
| | super family. | subfamily within the Ig super- |
| BOC | Brother of CDO. | family, consisting of an ectodomain |
| | | comprised of four (BOC) or five |
| | | (CDO) Ig repeats, followed by three |
| | | fibronectin type III (FNIII) repeats |
| | | and a long, divergent intracellular |
| | | domain. |
| GAS1 | Growth arrest specific | Regulates the amount of Hedgehog |
| | gene | ligand that can bind to Patched |
| | | receptors along with HHIP. |

II. Cytoplasmic Proteins

| | | |
|---|---|---|
| HFU | Human Fused | Stimulates GLI1 and GLI2 |
| | | transcription factors. |
| SUFU | Suppressor of fused | Sequesters GLI proteins in the |
| | homolog | cytoplasm and prevents |
| | | tarnscription of target genes. |
| STK36 | Serine/threonine-protein | Up-regulation of GLI transcription |
| | kinase 36 | activity. |
| ERK12* | Extracellular signal- | Up-regulation of GLI transcription |
| | regulated kinase | activity. |
| GLI1 | Transcriptional activator | Mediates target gene expression. |
| | Gli1 | |
| GLI2 | Transcriptional activator | Mediates target gene expression. |
| | Gli2 | |
| GLI3_R | Transcriptional repressor | Antagonises target gene expression |
| | Gli3 | by other Gli factors. |
| RAS* | Ras protein (GTPase | RAS and TWIST activate GLI1 |
| | activity) | regulatory sequences. |
| TWIST* | Twist-related protein | Is known to activate GLI1. |
| PKA_A | Protein Kinase alpha | Phosphorylates and activates SMO. |
| BTRCP | Beta-transducin repeat- | Involoved in ubiquitination of Gli1 |
| | containing protein | resulting in the formation of a |
| | | transcriptional repressor. |
| CKI_A | Casein Kinase I isoform | Known to elicit negative effects on |
| | alpha | GLI |
| GSK3 | Glycogen synthase | Known to elicit negative effects on |
| | Kinase 3 | GLI |
| NOTCH1* | Notch1 protein | Known to elicit negative effects on |
| | | GLI |
| FAS* | Apoptosis-mediating | Mediates apoptosis |
| | surface antigen FAS | |
| ULK3 | Unc-51-like kinase 3 | Serine Threonine kinase present in |
| | | addition to STK36 that functions in |
| | | up-regulation of GLI transcriptional |
| | | activity |

III. Nuclear Proteins

| | | |
|---|---|---|
| NUC_GLI1 | Nuclear GLI1 | Represents the nuclear form of |
| | | GLI1 protein. |
| NUC_GLI2 | Nuclear GLI2 | Represents the nuclear form of |
| | | GLI2 protein. |
| NUC_SUFU | Nuclear SUFU | Represents the nuclear form of |
| | | SUFU. |
| NUC_STK36 | Nuclear STK36 | Represents the nuclear form of |
| | | STK36 |
| GLI3_A | Activated GLI3 for | Mediates target gene expression |
| | Transcription | |
| SKI | Proto-oncogene C-Ski | Functions as a transcriptional co- |
| | | repressor |
| NCOR | Nuclear receptor | Functions as a transcriptional co- |
| | corepressor | repressor |
| HDAC | Histone deacetylase | Functions as a transcriptional co- |
| | | repressor |
| SNO | Ski-like protein or Ski- | Functions as a transcriptional co- |
| | related oncogene | repressor |
| SIN3A | Paired amphipathic helix | Functions as a transcriptional co- |
| | protein Sin3 alpha | repressor |
| DYRK1 | Dual Specificity | Known to substantially increase |
| | Tyrosine | GLI mediated transcription |
| | phosphorylation | |
| | Regulated Kinase 1A/ | |
| | Dual Specificity Yak1 | |
| | related Kinase | |
| NUMB | Protein numb homolog | Numb along with ubiquitin ligase |
| ITCH | E3 ubiquitin-protein | such as Itch is able to |
| | ligase Itchy homolog | polyubiquitinate GLI1 and target it |
| | | for degradation and thus control HH |
| | | signaling. |

IV. Output Proteins

| | | |
|---|---|---|
| CTNNB_TCF4 | Nuclear form of TCF4 | Represents the nuclear form of |
| | | TCF4 |
| CYCLIN_B | G2/mitotic-specific | Mediates cell cycle regulation |
| | cyclin-B1 | |
| CYCLIN_D | G1/S-specific cyclin-D | Mediates cell cycle regulation |
| CYCLIN_D2 | G1/S-specific cyclin-D2 | Mediates cell cycle regulation |
| CYCLIN_E | G1/S-specific cyclin-E | Mediates cell cycle regulation |
| FOXM1 | Forkhead box protein | Implicated in cellular proliferation |
| | M1 | |
| PDGFRA | Platelet Derived Growth | Transmembrane receptor |
| | Factor receptorisoform | |
| | alpha | |
| OPN | Osteopontin | Osteopontin is a secreted protein |
| | | that influences multiple downstream |
| | | signaling events that allow cancer |
| | | cells to resist apoptosis, evade host |
| | | immunity and influence growth of |
| | | indolent tumors. |
| CMYC | Myc proto-oncogene | Mediates cellular proliferation. |
| | protein | |
| BMI | Polycomb complex | BMI-1 is a transcriptional repressor |
| | protein BMI-1 | belonging to the polycomb gene |
| | | family and its suppressor functions |
| | | are involved in maintaining |
| | | neuronal, haematopoietic and |
| | | mammary gland stem cells. |
| SNAI1 | Protein snai1 homolog 1 | Responsible for the degradation of |
| | | E-cadherin and initiation of |
| | | invasion |
| JAGGED2 | Notch ligand Jagged | Stimulates Notch signaling |
| SFRP | Secreted frizzled-related | Wnt antagonist |
| | protein | |
| WNT | Wnt family proteins or | Representative of a WNT ligand |
| | ligand | |
| BCL2 | Apoptosis regulator Bcl-2 | Anti-apoptotic |

V. Cellular Responses

| | | |
|---|---|---|
| Anti_Apop | Anti apoptosis | These are the cellular responses or |
| Notch_Signal | Nocth signaling | phenotypic expressions that have |
| Wnt_Signal | Wnt signaling | been shown as outcomes of this |
| Cellcycle_Progression | Cell cycle progression | pathway. |
| Emt | Epithelial to | |
| | Mesenchymal transition | |
| Cell_Proliferation | Cellular proliferation | |

*These proteins do not belong to the core proteins of Hedgehog pathway. We have considered these proteins so as to include direct cross talks by other molecules of different pathway which may influence different cancer scenarios.

In the above table, the inventors have used the abbreviations of the proteins and cellular responses that are included in the constructed Hedgehog signaling pathway map. The entire table has been divided into five parts: I. Extracellular and Membrane Proteins. II. Cytoplasmic Proteins. III. Nuclear Proteins. IV. Output Proteins. V. Cellular Responses. The names of the proteins and corresponding documentation are taken from the databases listed in Table 1 and from the literatures. [11, 34-60].

TABLE 4
Logical States of the Input and Output proteins of Hedgehog signaling
in Normal, Glioma, Colon and Pancreatic Cancer Scenarios.

| Normal | | Glioma | |
|---|---|---|---|
| Input | Output | Input | Output |

| | | | | | | | |
|---|---|---|---|---|---|---|---|
| BMP_RUNX3 | 0 | DHH | 0 | SHH | 1 | DHH | 1 |
| DISPATCHED | 1 | IHH | 0 | DHH | 1 | IHH | 1 |
| HHAT | 1 | SHH | 1 | IHH | 1 | SHH | 1 |
| CDO | 1 | PTCH1 | 1 | BMP_RUNX3 | 1 | PTCH1 | 0 |
| BOC | 1 | PTCH2 | 0 | DISPATCHED | 1 | PTCH2 | 0 |
| GAS1 | 0 | SMO | 1 | HHAT | 1 | SMO | 0 |
| HFU | 0 | STK36 | 1 | CDO | 1 | STK36 | 0 |
| ULK3 | 0 | GLI1 | 1 | BOC | 1 | GLI1 | 0 |
| NOTCH1 | 0 | GLI2 | 1 | GAS1* | 0 | GLI2 | 0 |
| SUFU | 0 | NUC_GLI1 | 1 | GLI1 | 0 | NUC_GLI1 | 0 |
| TWIST | 0 | NUC_GLI2 | 1 | GLI2 | 0 | NUC_GLI2 | 0 |
| RAS | 0 | GLI3_A | 1 | HFU | 1 | GLI3_A | 0 |
| ERK12 | 0 | GLI3_R | 0 | ULK3 | 1 | GLI3_R | 1 |
| PKA_A | 0 | FAS | 0 | NOTCH1 | 0 | FAS | 1 |
| BTRCP | 0 | CYCLIN_B | 1 | SUFU* | 0 | CYCLIN_B | 0 |
| CKI_A | 0 | CYCLIN_D | 1 | TWIST | 1 | CYCLIN_D | 0 |
| GSK3 | 0 | CYCLIN_D2 | 1 | RAS | 1 | CYCLIN_D2 | 0 |
| DYRK1 | 1 | CYCLIN_E | 1 | ERK12 | 1 | CYCLIN_E | 0 |
| NUMB | 0 | FOXM1 | 1 | PKA_A* | 0 | FOXM1 | 0 |
| ITCH | 0 | PDGFRA | 1 | BTRCP* | 0 | PDGFRA | 0 |
| SKI | 0 | CTNNB_TCF4 | 0 | CKI_A* | 0 | CTNNB_TCF4 | 1 |
| NCOR | 0 | OPN | 1 | GSK3* | 0 | OPN | 0 |
| HDAC | 0 | CMYC | 1 | DYRK1 | 1 | CMYC | 0 |
| SNO | 0 | BMI | 1 | NUMB* | 0 | BMI | 0 |
| SIN3A | 0 | SNAI1 | 1 | ITCH* | 0 | SNAI1 | 0 |
| NUC_STK36 | 1 | JAGGED2 | 1 | SKI* | 0 | JAGGED2 | 0 |
| NUC_SUFU | 0 | SFRP | 1 | NCOR* | 0 | SFRP | 0 |
| NA | NA | WNT | 1 | HDAC* | 0 | WNT | 0 |
| NA | NA | BCL2 | 1 | SNO* | 0 | BCL2 | 0 |
| NA | NA | NA | NA | SIN3A* | 0 | HHIP | 1 |
| NA | NA | NA | NA | NUC_STK36 | 1 | NA | NA |
| NA | NA | NA | NA | NUC_SUFU* | 0 | NA | NA |

| Colon | | | | Pancreatic | | | |
|---|---|---|---|---|---|---|---|
| Input | | Output | | Input | | Output | |
| SHH | 1 | DHH | 0 | DHH | 1 | DHH | 1 |
| IHH | 1 | SHH | 1 | SHH | 1 | SHH | 1 |
| BMP_RUNX3 | 1 | IHH | 1 | IHH | 1 | IHH | |
| DISPATCHED | 1 | PTCH1 | 1 | BMP_RUNX3 | 1 | PTCH1 | 1 |
| HHAT | 1 | PTCH2 | 0 | DISPATCHED | 1 | PTCH2 | 0 |
| CDO | 1 | SMO | 1 | HHAT | 1 | SMO | 1 |
| BOC | 1 | STK36 | 1 | CDO | 1 | STK36 | 1 |
| GAS1 | 0 | GLI1 | 1 | BOC | 1 | GLI1 | 1 |
| HFU | 1 | GLI2 | 1 | GAS1 | 0 | GLI2 | 1 |
| ULK3 | 1 | NUC_GLI1 | 1 | HFU | 1 | NUC_GLI1 | 1 |
| NOTCH1 | 0 | NUC_GLI2 | 1 | ULK3 | 1 | NUC_GLI2 | 1 |
| SUFU* | 0 | GLI3_A | 1 | NOTCH1 | 0 | GLLI3_A | 1 |
| TWIST | 0 | GLI3_R | 0 | SUFU* | 0 | GLI3_R | 0 |
| RAS | 1 | FAS | 0 | TWIST | 0 | FAS | 0 |
| ERK12 | 0 | CYCLIN_B | 1 | RAS | 1 | CYCLIN_B | 1 |
| PKA_A* | 0 | CYCLIN_D | 1 | ERK12 | 1 | CYCLIN_D | 1 |
| BTRCP* | 0 | CYCLIN_D2 | 1 | PKA_A* | 0 | CYCLIN_D2 | 1 |
| CKI_A* | 0 | CYCLIN_E | 1 | BTRCP* | 0 | CYCLIN_E | 1 |
| GSK3* | 0 | FOXM1 | 1 | CKI_A* | 0 | FOXM1 | 1 |
| DYRK1 | 1 | PDGFRA | 1 | GSK3* | 0 | PDGFRA | 1 |
| NUMB* | 0 | CTNNB_TCF4 | 0 | DYRK1 | 1 | CTNNB_TCF4 | 0 |
| ITCH* | 0 | OPN | 1 | NUMB* | 0 | OPN | 1 |
| SKI* | 0 | CMYC | 1 | ITCH* | 0 | CMYC | 1 |
| NCOR* | 0 | BMI | 1 | SKI* | 0 | BMI | 1 |
| HDAC* | 0 | SNAI1 | 1 | NCOR* | 0 | SNAI1 | 1 |
| SNO* | 0 | JAGGED2 | 1 | HDAC* | 0 | JAGGED2 | 1 |
| SIN3A* | 0 | SFRP | 1 | SNO* | 0 | SFRP | 1 |
| NUC_STK36 | 1 | WNT | 1 | SIN3A* | 0 | WNT | 1 |
| NUC_SUFU* | 0 | BCL2 | 1 | NUC_STK36 | 1 | BCL2 | 1 |
| NA | NA | HHIP | 1 | NUC_SUFU* | 0 | NA | NA |
| NA | NA | NA | NA | NA | NA | NA | NA |
| NA | NA | NA | NA | NA | NA | NA | NA |

N/A: Not Applicable
*Proteins having Loss of Function in cancer scenario.

This table shows the Logical states used in CellNetAnalyzer [23] for simulating the Hedgehog Pathway model of Normal, Glioma, Colon and Pancreatic cancer scenarios. The logical states of the input proteins for each scenario and the respective simulated results of the output proteins are given in this table. The logical state ‘1’ of a node represents its expression or as “ON” state where “0” represents the “OFF” state. The Logical states of the input proteins were considered from various database (See Table 1) and literature sources [11, 19, 32-35, 50, 54, 60]. In the present invention, only the simulation results of “Time scale 2” are described for each scenario.

TABLE 5
Boolean equations with the documentation for the Hedgehog pathway

| Interactions | Documentation |
|---|---|
| →DISPATCHED | Inputs to the model. Upstream regulators of |
| →HHAT | these molecules have not been considered. |
| → CDO | |
| → BOC | |
| → NUC_SUFU | |
| → GAS1 | |
| →BMP_RUNX3 | |
| →ULK3 | |
| → HFU | |
| → SUFU | |
| → ERK12 | |
| → RAS | |
| → TWIST | |
| → DYRK1 | |
| → NUMB | |
| → ITCH | |
| → PKA_ALPHA | |
| → BTRCP | |
| → CKI_A | |
| → GSK3 | |
| → NUC_STK36 | Inputs to the model. Upstream regulators of |
| → NOTCH1 | these molecules have not been considered. |
| → SKI | |
| → SNO | |
| → NCOR | |
| → SIN3 ALPHA | |
| → HDAC | |
| BMP_RUNX3→IHH | BMP-RUNX3 signaling induces expression of |
| | IHH in surface differentiated epithelial cells of |
| | stomach and intestine [11]. |
| CDO + BOC→SHH | CDO and BOC bind SHH through a high- |
| | affinity interaction with a specific fibronectin |
| | repeat that is essential for activity. They |
| | demonstrate that CDO and BOC are necessary |
| | but not sufficient for activation [34]. However, |
| | there is no evidence for the exact mechanism |
| | and if both are required for the enhancement of |
| | signaling. |
| DISPATCHED + HHAT + !HHIP→DHH | Dispatched regulates the release and |
| | extracellular accumulation of cholesterol- |
| | modified hedgehog proteins and is hence |
| | required for effective production of the HH |
| | signal [35], [36]. HHAT (Hedgehog |
| | acyltransferase) is a hedgehog modifier which |
| | induces lipid modification to generate mature |
| | peptides. HH proteins with lipid modification |
| | are then released from producing cells by |
| | Dispatched homologues [11]. HHIP can |
| | antagonize all types of HH ligands [37]. |
| DISPATCHED + HHAT + !HHIP→IHH | Dispatched and HHAT system also operates in |
| | the same way as during DHH release [35], |
| | [36]. HHIP can antagonize all types of HH |
| | ligands [37]. |
| DISPATCHED + HHAT + !HHIP + !GAS1→SHH | Dispatched and HHAT also operate in the |
| | same way as during DHH release [35], [36]. |
| | HHIP is found to bind directly to SHH and |
| | attenuate SHH signaling like PTCH1/2 while |
| | its expression was induced by SHH signals |
| | [37], [38]. |
| !DHH + !IHH + !SHH→PTCH1_Free | Negative influence of all the Hedgehog ligands |
| !DHH + !IHH + !SHH→PTCH2_Free | was considered to denote the inactive state of |
| | Patched (PTCH1 and PTCH2) receptors. In the |
| | absence of Hedgehog ligands Patched |
| | receptors are active and suppress the activity of |
| | Smoothened [39]. |
| DHH + !PTCH1_Free→SMO | In the absence of a stimulus by Hedgehog, |
| IHH + !PTCH1_Free→SMO | Patched receptor inhibits Smoothened. Upon |
| SHH + !PTCH1_Free→SMO | binding of Hedgehog ligands DHH, SHH or |
| SHH + !PTCH2_Free→SMO | IHH to Patched, Smoothened is activated |
| | leading to the transcription of target genes. |
| | This is also reported that mutations affecting |
| | the transmembrane proteins Patched or |
| | Smoothened trigger the ligand independent |
| | activity of Hedgehog signaling pathway and |
| | are hence associated with human tumors such |
| | as basal cell carcinoma and medulloblastoma |
| | [39]. |
| SMO→STK36 | SMO binds to STK36 to stabilize GLI proteins |
| | [11]. |
| !SMO→FAS | SMO expression inhibits FAS thereby |
| | preventing apoptosis [40]. |
| HFU + !PKA_A + !GSK3 + | HFU enhances GLI1 function in a manner that |
| !CKI_A + !BTRCP + !SUFU→GLI1 | is independent of a functional kinase domain |
| | [32]. GSK3 phosphorylates GLI proteins post |
| | phosphorylation by PKA and it is known to |
| | elicit negative effects [33]. SMO inactivation |
| | leads to formation of the cytoplasmic GLI |
| | degradation complex, in which GLI family |
| | members (GLI1, GLI2 and GLI3) are |
| | phosphorylated by casein kinase alpha |
| | (CKI_α), glycogen synthase kinase-3β |
| | (GSK3β) and protein kinase A (PKA). |
| | Phosphorylated GLI is recognized by |
| | FBXW1/BTRCP1 and FBXW11/BTRCP2 for |
| | ubiquitination, and ubiquitinated GLI is |
| | partially degraded to release its intact N- |
| | terminal half thereby functioning as |
| | transcriptional repressor [11]. The inhibitory |
| | interactions have been included with activation |
| | interactions using an AND operator. Therefore |
| | GLI cannot be activated unless and until all the |
| | inhibitors are absent. However, this needs to be |
| | checked in in vivo conditions. But in the model |
| | it is necessary to introduce these interactions |
| | using an “AND” operation to assure signal |
| | flow. |
| ERK12 + !PKA_A + !GSK3 + | EGFR signals via ERK potentiate target gene |
| !CKI_A + !BTRCP + !SUFU→GLI1 | activation via GLI1 [41]. |
| RAS + !PKA_A + !GSK3 + | It is reported that oncogenic KRAS/ |
| !CKI_A + !BTRCP + !SUFU→GLI1 | constitutively active RAS in Pancreatic Cancer |
| | cells, increases the transcription of GLI1 levels |
| | [42]. |
| TWIST + !PKA_A + !GSK3 + | TWIST activates human GLI1 regulatory |
| !CKI_A + !BTRCP + !SUFU→GLI1 | sequences via two E-boxes in GLI1's first |
| | intron. Demonstrated in a murine model and |
| | using human GLI sequences. Two critical cis |
| | elements in human GLI1 gene: a GC box that |
| | binds Sp1 at 195 and two E-boxes that operate |
| | at 157 and 482 have also identified. The 157 E- |
| | box binds USF1 and USF2, while E-box 482 |
| | binds TWIST. Sp1 and USf1/2 are |
| | ubiquitiously expressed TFs and can function |
| | either as activators or repressors depending |
| | upon cellular context. However their roles have |
| | not been clearly delineated and hence not |
| | incorporated in the model [43]. |
| ULK3 + !PKA_A + !GSK3 + | ULK3, a Ser/Threonine kinase present in |
| !CKI_A + !BTRCP + !SUFU→GLI1 | addition to STK36 is essential for the up- |
| | regulation of GLI transcriptional activity. It |
| | phosphorylates GLI1 in both N (1-426) and C |
| | (754-1126) terminus but the fragment of gli1 |
| | between residues 426-754 is not |
| | phosphorylated by ULK3. Thus ULK3 is a |
| | positive activator [33]. |
| STK36 + !PKA_A + !GSK3 + | STK36 is a positive regulator of SHH pathway |
| !CKI_A + !BTRCP + !SUFU→GLI1 | that acts independent on is functional kinase |
| | domain. STK36 enhances GLI2 activity but not |
| | GLI1 in C3H10T1/2 and HEK293 cells and |
| | Gli1 transcriptional activity in NIH3T3C2. |
| | Sn480 cells. Hence STK36 expression is cell |
| | type specific. As ours is a master model we |
| | have nevertheless included this interaction |
| | [33]. |
| GLI2 + !PKA_A + !GSK3 + | GLI1 is a direct target of GLI2. The study was |
| !CKI_A + !BTRCP + !SUFU→GLI1 | conducted in normal human epidermis and |
| | Basal cell carcinoma cells [44]. |
| GLI3_A + !SKI + !SNO + | GLI3 exists in two forms - a full-length |
| !NCOR + !SIN3A + !HDAC→GLI1 | transcriptional activator (GLI3A) or an amino- |
| | terminal fragment that functions as a repressor. |
| | This particular activator isoform is GLI3A |
| | [45]. On the other hand SKI, SKI related |
| | protein SNO, NCOR, SIN3A, HDACs form a |
| | transcriptional repressor complex that interfere |
| | with GLI1 activation by full length GLI3 [46]. |
| | Hence they are included in the AND |
| | interaction. |
| GLI1→FOXM1 | FOXM1/FOXL1 is a direct target of GLI |
| | mediated activation [11]. |
| FOXM1→CELL_PROLIFERATION | FOXM1 is known to mediate cell proliferative |
| | functions [47]. |
| CELL_PROLIFERATION→ | Cellular response in our model. |
| !GLI1→GLI3_R | Repressor form of GLI3 produced in the |
| | absence of GLI1 expression [19]. |
| ! GLI3_R → GLI3_ACTIVE | GLI3_ACTIVE form is produced in the |
| | absence of GLI3 repressor form [19] |
| GLI1→PDGFRA | PDGFRA is expressed at high levels in human |
| PDGFRA→ | and murine Basal Cell Carcinoma. It has been |
| | found that ectopic expression of GLI increases |
| | PDGFRA levels i.e. increases receptor protein |
| | levels whereas inhibition of the HH pathway |
| | reduces PDGFRA levels [48]. |
| GLI1→NUC_GLI1 | Cellular location of GLI1 has been found in |
| | Cytoplasm as well as in Nucleus [35]. We |
| | considered this transportation in our model and |
| | named NUC_GLI1 of the nuclear counterpart |
| | of GLI1. |
| NUC_GLI1 + NUC_STK36 + DYRK1 + | PTCH1 and HHIP receptors inhibit the |
| !NUC_SUFU + !NUMB + !ITCH→PTCH1 | pathway in the absence of a stimulus or |
| NUC_GLI1 + NUC_STK36 + DYRK1 + | Hedgehog ligands. This indicates the formation |
| !NUC_SUFU + !NUMB + !ITCH→HHIP | of a negative feedback loop [48]. DYRK1 |
| | (Dual Specificity Tyrosine phosphorylation |
| | Regulated Kinase 1A/Dual Specificity Yak1 |
| | related Kinase) can substantially enhance GLI1 |
| | dependent transcription. It has also been |
| | suggested that failure of SHH to stimulate |
| | DYRK1 kinase activity is indicative to the fact |
| | that DYRK1 may not be regulated by the SHH |
| | signaling pathway but functionally interacts |
| | with it [49]. SUFU inhibits the activator |
| | isoform of GLI proteins and activates the |
| | repressor forms [41]. A proper balance |
| | between both the forms regulates Wnt |
| | signaling. It is also reported that NUMB along |
| | with ubiquitin ligase such as ITCH is able to |
| | polyubiquitinate Gli1 and target it for |
| | degradation and thus control HH signaling. |
| | Thus all these components have been included |
| | in the AND equation as they affect the final |
| | outcome. |
| NUC_GLI1 + NUC_STK36 + DYRK1 + | It is reported GLI1 is also produced at the end |
| !NUC_SUFU + !NUMB + !ITCH→GLI1 | of this pathway and thus create a positive |
| | feedback loop [50], [51]. |
| NUC_GLI1 + NUC_STK36 + DYRK1 + | Osteopontin (OPN) is a direct transcriptional |
| !NUC_SUFU + !NUMB + !ITCH→OPN | target of GLI1 demonstrated in MDA-MB 435 |
| OPN → | cell line. OPN is a secreted protein that |
| | influences multiple downstream signaling |
| | events that allow cancer cells to resist |
| | apoptosis, invade through ECM, evade host |
| | immunity and influence growth of indolent |
| | tumors. OPN is expressed by normal cells. |
| | However sustained expression in cancer cells |
| | promotes aberrant growth of cells and an |
| | invasive phenotype [52]. |
| NUC_GLI1 + NUC_STK36 + DYRK1 + | Hedgehog signaling regulates the proliferation |
| !NUC_SUFU + !NUMB + !ITCH→CYCLIN_D | of distinct cell types via direct activation of |
| NUC_GLI1 + NUC_STK36 + DYRK1 + | genes that are involved in cell cycle |
| !NUC_SUFU + !NUMB + !ITCH→CYCLIN_E | progression and mediate G1 to S transition. |
| CYCLIN_D→CELLCYCLE_PROGRESSION | Cyclins such as CYCLIN D and CYCLIN E |
| CYCLIN_E→CELLCYCLE_PROGRESSION | are involved in regulation of cell cycle. |
| CELLCYCLE_PROGRESSION→ | Cell cycle progression is one of the cellular |
| | responses that have been considered in our |
| | model. |
| NUC_GLI1 + NUC_STK36 + DYRK1 + | Expression of GLI1 and C-MYC has also |
| !NUC_SUFU + !NUMB + !ITCH→CMYC | found in various experiments [52], [53]. |
| CMYC→ | |
| NUC_GLI1 + NUC_STK36 + DYRK1 + | Hedgehog signalling leads to an increased |
| !NUC_SUFU + !NUMB + !ITCH→BMI | expression of BMI-1 in isolated mammary |
| | epithelial stem cells and CSCs. BMI-1 is a |
| | transcriptional repressor belonging to the |
| | polycomb gene family and its suppressor |
| | functions are involved in maintaining neuronal, |
| | haematopoietic and mammary gland stem cells. |
| | It leads to self renewal [41]. Activated STK36 |
| | also phosphorylates SUFU to promote the |
| | nuclear accumulation of full length GLI [11]. It |
| | is also reported that activation of hedgehog |
| | signaling increases mammosphere- initiating |
| | cell number and mammosphere size, whereas |
| | inhibition of the pathway results in a reduction |
| | of these effects. These effects are mediated by |
| | the polycomb gene bmi [55]. |
| BMI→ | Output protein considered in our model. |
| NUC_GLI1 + NUC_STK36 + DYRK1 + | It is reported that activation of SNAI1 a protein |
| !NUC_SUFU + !NUMB + !ITCH→SNAI1 | responsible for degradation of cadherin and |
| SNAI1→EMT | induction of invasion is directly activated by |
| | GLI1 [41]. SNAI1 protein is responsible for |
| | epithelial to mesenchymal transition. |
| EMT→ | Cellular response Epithelial to Mesenchymal |
| | Transition. |
| NUC_GLI1 + NUC_STK36 + DYRK1 + | Expression of JAGGED2 has been reported |
| !NUC_SUFU + !NUMB + !ITCH→JAGGED2 | [50]. |
| JAGGED2→NOTCH_SIGNAL | JAGGED2 is a notch, ligand, hence promotes |
| | notch signaling. |
| NOTCH_SIGNAL→ | Cellular response. |
| NUC_GLI1 + NUC_STK36 + DYRK1 + | Expression of SFRP by GLI1 has been found |
| !NUC_SUFU + !NUMB + !ITCH→SFRP | [50], [56]. |
| NUC_GLI1 + NUC_STK36 + DYRK1 + | GLI1 mediates the activation of wnt family |
| !NUC_SUFU + !NUMB + !ITCH→WNT | proteins and enhances signaling via these |
| | pathways, exact wnt ligand is not known and |
| | thus not included in our model [19]. |
| WNT + !SFRP→WNT_SIGNAL | Activation of Wnt signaling depends on the |
| | presence of WNT ligand and absence of its |
| | antagonist SFRP [57]. |
| WNT_SIGNAL→ | Cellular response. |
| NUC_GLI1 + NUC_STK36 + DYRK1 + | In epidermal cells GLI1 can induce the |
| !NUC_SUFU + !NUMB + !ITCH→BCL2 | expression of antiapoptotic factor BCL2 [41]. |
| HFU + !PKA_A + !GSK3 + | HFU enhances GLI2 function in a manner that |
| !CKI_A + !BTRCP + !SUFU→GLI2 | is independent of a functional kinase domain |
| | [32]. |
| STK36 + !PKA_A + !GSK3 + | STK36 is a positive regulator of GLI2 activity |
| !CKI_A + !BTRCP + !SUFU→GLI2 | [33]. STK36 enhances GLI2 activity but not |
| | GLI1 in C3H10T1/2 and HEK293 cells. GSK3 |
| | phosphorylates GLI proteins post |
| | phosphorylation by PKA and it is known to |
| | elicit negative effects. On the other hand SMO |
| | inactivation leads to formation of the |
| | cytoplasmic GLI degradation complex, in |
| | which GLI family members (GLI1, GLI2 and |
| | GLI3) are phosphorylated by casein kinase I |
| | (CKI), glycogen synthase kinase-3B (GSK3β) |
| | and protein kinase A (PKA) [11]. |
| | Phosphorylated GLI is recognized by |
| | FBXW1/BTRCP1 and FBXW11/BTRCP2 for |
| | ubiquitination, and ubiquitinated GLI is |
| | partially degraded to release its intact N- |
| | terminal half functioning as transcriptional |
| | repressor. Thus all the above factors are |
| | included in the AND equation as they |
| | influence the formation of GLI2 |
| HFU + !NOTCH1→GLI2 | HFU enhances GLI2 function in a manner that |
| | is independent of a functional kinase domain |
| | [32]. Inactivation of notch1 gene in epidermis |
| | induces sustained expression of GLI2 and |
| | causes Basal Cell Carcinoma [48]. Thus, GLI2 |
| | is activated in the presence of HFU and |
| | absence of notch1. This interaction has been |
| | represented as an AND equation. |
| STK36 + !NOTCH1→GLI2 | We have represented an alternative mode of |
| | GLI2 activation by STK36 without the |
| | presence of NOTCH1. |
| GLI2→NUC_GLI2 | GLI2 when transported to the nucleus is |
| | represented as NUC_GLI2. |
| NUC_GLI2 + NUC_STK36 + !NUC_SUFU→CYCLIN_D2 | However, this interaction has been found in a |
| | murine model [41], [58], we included this |
| | interaction as CYCLIN_D2 is one of the |
| | important proteins for cellcycle progression. |
| | Activated STK36 also phosphorylates SUFU to |
| | promote the nuclear accumulation of full |
| | length GLI. |
| CYCLIN_D2→CELLCYCLE_PROGRESSION | CYCLIN D2 is implicated in cell cycle |
| | regulation. |
| NUC_GLI2 + NUC_STK36 + !NUC_SUFU→BCL2 | Epidermal cells GLI2 can induce the |
| | expression of anti apoptotic factor BCL2 [41]. |
| BCL2→ANTI_APOP | BCL2 is a known anti-apoptotic factor [59]. |
| ANTI_APOP→ | Cellular responses. |
| !GLI1 → CTNNB_TCF4 | Coincident of high-to-low TCF and low-to- |
| CTNNB_TCF4 → | high HH-GLI1 transitions in patient Colon |
| | Carcinoma have been found. Therefore, we can |
| | write that higher level of expression of GLI1 |
| | inhibits the activity of TCF complex [61]. |
| GLI3_A + NUC_STK36 + !NUC_SUFU→CYCLIN_D2 | Similarly active form of GLI3 mediates the |
| | activation of CYCLIN_D2. |
| !PTCH1→CYCLIN_B | It is reported that PTCH regulates the activity |
| | of CYCLIN_B. Interaction with Patched in the |
| | cytoplasm blocks cell proliferation by |
| | preventing nuclear localization of the activated |
| | complex. Ligand induced activation of this |
| | complex leads to the nuclear localization of |
| | CYCLIN B by disruption of the physical |
| | interaction between Patched 1 and CYCLIN B |
| | [48]. |
| CYCLIN_B→CELLCYCLE_PROGRESSION | CYCLIN B is also implicated in cell cycle |
| | regulation. |

TABLE 6
Comparison of the percentage of accuracy between experimental
and simulation results for each cancer scenario

| | | Not | | | |
|---|---|---|---|---|---|
| | | Determined/ | Correct | Incorrect | |
| | | Not | Predic- | Predic- | *Accuracy |
| Disease | Comparison | Available | tions | tions | (in %) |
| Glioma | SIM1 with | 24 | 22 | 11 | 66.66 |
| | EXP | | | | |
| | SIM2 with | 0 | 54 | 3 | 94.37 |
| | SIM1 | | | | |
| | SIM2 with | 24 | 25 | 8 | 75.75 |
| | EXP | | | | |
| Colon | SIM1 with | 52 | 5 | 0 | 100.00 |
| Cancer | EXP | | | | |
| | SIM2 with | 0 | 57 | 57 | 100.00 |
| | SIM1 | | | | |
| | SIM2 with | 52 | 5 | 0 | 100.00 |
| | EXP | | | | |
| Pancreatic | SIM1 with | 13 | 25 | 19 | 56.80 |
| Cancer | EXP | | | | |
| | SIM2 with | 0 | 47 | 10 | 82.45 |
| | SIM1 | | | | |
| | SIM2 with | 13 | 32 | 12 | 72.72 |
| | EXP | | | | |

*Accuracy (%) = ((Correct Predictions)/(Correct Predictions) + (Incorrect Predictions)) × 100 (%)

TABLE 7
Significant proteins extracted from Connectivity analysis

| | Extracellular | | | Output |
|---|---|---|---|---|
| Parameters | and Ligands | Cytoplasm | Nucleus | proteins |
| In-Degree | DHH(3), IHH(4), | GLI1(24), | NOT FOUND | [OPN, CYCLIN_D, |
| (>2.45) | SHH(6), PTCH1(9), | GLI2(8) | | CYCLIN_E, CMYC, |
| | PTCH2(3), HHIP(6) | | | BMI, SNAI1, |
| | | | | JAGGED2, SFRP, |
| | | | | WNT](6) |
| | | | | BCL2(7), CYCLIN_D2[4] |
| Out-Degree | DISPATCHED(3), | GLI1(6) | [DYRK1, NUMB, | NOT FOUND |
| (>2.45) | HHAT(3), HHIP(3) | | ITCH, NUC_GLI1] | |
| | | | (13), NUC_STK36 | |
| | | | (14), NUC_SUFU (14) | |
| Total- | DHH(5), IHH(6), | GLI1(30), | [DYRK1, NUMB, | [OPN, CYCLIN_D, |
| Degree | SHH(8), PTCH1(11), | GLI2(10) | ITCH](13), | CYCLIN_E, CMYC, |
| (>4.91) | HHIP(9) | | [NUC_GLI1, | BMI, SNAI1, |
| | | | NUC_SUFU, | JAGGED2, SFRP, |
| | | | NUC_STK36] (14) | WNT](6), BCL2(7) |

### REFERENCES

1. Dillon R, Gadgil C, Othmer H G (2003) Short- and long-range effects of Sonic hedgehog in limb development. Proc. Natl. Acad. Sci. 100(18): 10152-10157.
2. Saha K, Schaffer D V (2006) Signal dynamics in Sonic hedgehog tissue patterning.
Development 1411:889-900.
3. Yiwei Li, Ma'in Y Maitah, Aamir Ahmad, Dejuan Kong, Bin Bao (2012) Targeting the Hedgehog signaling pathway for cancer therapy. Expert Opin. Ther. Targets 16(1):49-66.
4. Kenneth Paul Olive, David Tuveson (2012) Hedgehog Pathway Inhibitors. United States Patent Application Publication. Publication number: US 2012/0020876 A1.
5. Scott X. Atwood, Anne Lynn S. Chang, Anthony E. Oro (2012) Hedgehog pathway inhibition and the race against tumor evolution. J. Cell Biol 199(2):193-197.
6. Charles M. Rudin, Christine L. Hann, John Laterra, Robert L. Yauch, Christopher A. Callahan et. al. (2009) Treatment of Medulloblastoma with Hedgehog Pathway Inhibitor GDC-0449. The New England Journal Of Medicine 361:1173-8.
7. Chris Dockendorff, Marek M. Nagiec, Michel Weïwer, Sara Buhrlage, Amal Ting et. al. (2012) Macrocyclic Hedgehog Pathway Inhibitors: Optimization of Cellular Activity and Mode of Action Studies. ACS Med. Chem. Lett. 3: 808-813.
8. Sherriff M R, Sarkar R R (2008) Computational approaches and modeling of signaling processes in the Immune System. Proc Indian Natn Sci Acad 74: 187-200.
9. Albert R, Othmer H G (2003) The topology of the regulatory interactions predicts the expression pattern of the segment polarity genes in Drosophila melanogaster. Journal of Theoretical Biology 223:1-18.
10. Subramanian K, Gadgil C. (2009) Robustness of the Drosophila segment polarity network to transient perturbations. Chemical Engineering 1-13.
11. Katoh Y, Katoh M (2006) Hedgehog signaling pathway and gastrointestinal stem cell signaling network (review). International Journal of Molecular Medicine 18:1019-23.
12. Saitoh Y, Kuratsu J, Takeshima H, Yamamoto S, Ushio Y (1995) Expression of osteopontin in human glioma. Its correlation with the malignancy. Lab Invest; 72(1):55-63.
13. Btischges R, Weber R G, Actor B, Lichter P, Collins V P, Reifenberger G (1999) Amplification and expression of cyclin D genes (CCND1, CCND2 and CCND3) in human malignant gliomas. Brain Pathology, 9(3):435-42
14. Xiang Zhang, Ming Zha, An-yang Huang, Zhou Fei, Wei Zhang, Xi-ling Wang (2005) The effect of cyclinD expression on cell proliferation in human gliomas. Journal of Clinical Neuroscience, Volume 12, Issue 2, Pages 166-168.
15. Arnab Chakravarti, Meaghan A. Delaney, Elizabeth Noll, Peter McL. Black, Jay S. Loeffler et al. (2001) Prognostic and Pathologic Significance of Quantitative Protein Expression Profiling in Human Gliomas. Clin Cancer Res; 7:2387-2395.
16. Jakub Godlewski, Michal O. Nowicki, Agnieszka Bronisz, Shante Williams, Akihiro Otsuki et al. (2008) Targeting of the Bmi-1 Oncogene/Stem Cell Renewal Factor by MicroRNA-128 Inhibits Glioma Proliferation and Self-Renewal. Cancer Res; 17. Sung-Pil Han, Ji-Hoon Kim, Myoung-Eun Han, Hey-Eun Sim, Ki-Sun Kim (2011) SNAI1 is Involved in the Proliferation and Migration of Glioblastoma Cells. Cell Mol Neurobiol; 31:489-496.
18. Masaru Katoh (2007) Networking of WNT, FGF, Notch, BMP, and Hedgehog Signaling Pathways during Carcinogenesis. Stem Cell Rev; 3:30-38, DOI 10.1007/s12015-007-0006-6
19. Ariel Ruiz i Altaba, Pilar Sanchez, Nadia Dahmane (2002) Gli and Hedgehog in Cancer: Tumours, Embryos and Stem cells. Nature Reviews, Volume 2, pp 361-372.
20. Jeffrey Trent, Paul Meltzer, Mark Rosenblum, Griffith Harsh, Kenneth Kinzler et al. (1986) Evidence for rearrangement, amplification, and expression of c-myc in a human glioblastoma. Proc. Natl. Acad. Sci. USA, Vol. 83, pp. 470-473.
21. Gerhard Regl, Maria Kasper, Harald Schnidar, Thomas Eichberger, Graham W. Neill (2004) Activation of the BCL2 Promoter in Response to Hedgehog/GLI Signal Transduction Is Predominantly Mediated by GLI2. Cancer Res, 64; 7724. 22. Georg Feldmann, Surajit Dhara, Volker Fendrich, Djahida Bedja, Robert Beaty (2007) Blockade of Hedgehog Signaling Inhibits Pancreatic Cancer Invasion and Metastases: A New Paradigm for Combination Therapy in Solid Cancers. Cancer Research, 67:2187-2196, 23. Steffen Klamt, Axel von Kamp (2011) An application programming interface for CellNetAnalyzer. BioSystems, DOI: 10.1016/j.biosystems. 2011.02.002.
24. Lixin Sun, A1-Min Hui, Qin Su,1 Alexander Vortmeyer, Yuri Kotliarov, Sandra Pastorino et al. (2006) Neuronal and glioma-derived stem cell factor induces angiogenesis within the brain. Cancer Cell Vol. 9: 287-300.
25. Yue-Hong Bian, Shu-Hong Huang, Ling Yang, Xiao-Li Ma, Jing-Wu Xie et al. (2007) Sonic hedgehog-Gli1 pathway in colorectal adenocarcinomas. World J Gastroenterol; Vol. 13(11): 1659-166
26. Huadong Pei, Liang Li, Brooke L. Fridley, Gregory D. Jenkins, Krishna R. Kalari et al. (2009) FKBP51 Affects Cancer Cell Response to Chemotherapy by Negatively Regulating Akt. Cancer Cell Vol. 16: 259-266. 27. Funahashi, A., Tanimura, N., Morohashi, M., and Kitano, H., (2003) CellDesigner: a process diagram editor for gene-regulatory and biochemical networks, BIOSILICO, 1:159-162., 28. Mingguang Liu, Bingbing Dai, Shin-Hyuk Kang, Kechen Ban, Feng-Ju Huang et al. (2006) FoxM1B Is Overexpressed in Human Glioblastomas and Critically Regulates the Tumorigenicity of Glioma Cells. Cancer Res; 66:3593-3602.
29. Heikki Joensuu, Marjut Puputti, Harri Sihto, Olli Tynninen, Nina N Nupponen (2005) Amplification of genes encoding KIT, PDGFRα and VEGFR2 receptor tyrosine kinases is frequent in glioblastoma multiforme. The Journal of Pathology., 30. Sandra Reichratha, Cornelia S. L. Müllerb, Beate Gleissnera, Michael Pfreundschuha, Thomas Vogt (2010) Notch- and vitamin D signaling in 1,25(O H)2D3-resistant glioblastoma multiforme (GBM) cell lines. The Journal of Steroid Biochemistry and Molecular Biology; Volume 121, Issues 1-2, Pages 420-424.
31. Roth W, Wild-Bode C, Platten M, Grimmel C, Melkonyan H S (2000) Secreted Frizzled-related proteins inhibit motility and promote growth of human malignant glioma cells. Oncogene, 19(37):4210-20.
32. Østerlund T, Kogerman P (2006). Hedgehog signaling: how to get from Smo to Ci and Gli. Trends in Cell Biology 16(4):176-18033. Maloverjan A, Piirsoo M, Michelson P, Kogerman P and Østerlund T (2009). Identification of a novel serine/threonine kinase ULK3 as a positive regulator of Hedgehog pathway. Exp Cell Res 316(4):627-637
34. Tenzen T., Allen B. L., Cole F., Kang J. S., Krauss R. S., McMahon A. P. (2006) The cell surface membrane proteins Cdo and Boc are components and targets of the Hedgehog signaling pathway and feedback network in mice. Dev Cell 10(5):647-656. 35. The UniProt Consortium (2012) Reorganizing the protein space at the Universal Protein Resource (UniProt). Nucleic Acids Res. 40: D71-D75.
36. Evangelista M, Tian H, de Sauvage F J (2006) The hedgehog signaling pathway in cancer.
Clin. Cancer Res. 12(20 Pt 1):5924-59288.
37. Olsen C L, Hsu P P, Glienke J, Rubanyi G M, Brooks A R (2004) Hedgehog-interacting protein is highly expressed in endothelial cells but down-regulated during angiogenesis and in several human tumors. BMC Cancer 4:43: 1471-2407.
38. Tojo M, Kiyosawa H, Iwatsuki K, Kaneko F (2002) Expression of a sonic hedgehog signal transducer, hedgehog-interacting protein, by human basal cell carcinoma. British Journal of Dermatology 146(1):69-73.
39. Taipale J, Cooper M K, Maiti T, Beachy P A (2002) Patched acts catalytically to suppress the activity of Smoothened. Nature 418:892-896. 40. Athar M, Li C, Tang X, Chi S, Zhang X et al. (2004) Inhibition of smoothened signaling prevents ultraviolet B-induced basal cell carcinomas through regulation of Fas expression and apoptosis. Cancer Res 64:7545-7552
41. Kasper M, Jaks V, Fiaschi M, Toftgard R (2009) Hedgehog signalling in breast cancer. Carcinogenesis, 30(6):903-11. 42. Ji Z, Mei F C, Xie J, Cheng X (2007) Oncogenic KRAS activates hedgehog signaling pathway in pancreatic cancer cells. Jour Biol Chem 282(19):14048-5543. Villavicencio E H, Yoon J W, Frank D J, Ftichtbauer E M, Walterhouse D O et al.,(2002) Cooperative E-Box Regulation of Human GLI1 by TWIST and USF. Genesis 32(4):247-258.
44. Ikram M S, Neill G W, Regl G, Eichberger T, Frischauf A M et al. (2004) GLI2 is expressed in normal human epidermis and BCC and induces GLI1 expression by binding to its promoter. Journal of Investigative Dermatology; 122(6):1503-9.
45. Rohatgi R, Scott M P (2007) Patching the gaps in Hedgehog signalling. Nat Cell Biol 9(9):1005-1009.
46. Dai P, Shinagawa T, Nomura T (2002) Ski is involved in transcriptional regulation by the repressor and full-length forms of Gli3. Genes & Dev. 16:2843-2848.
47. Gong A, Huang S (2012) FoxM1 and Wnt/β-Catenin Signaling in Glioma Stem Cells. Cancer Res; 72(22):5658-62.
48. Pasca M M, Hebrok M (2003) Hedgehog signalling in cancer formation and maintenance. Nat Rev Cancer 3(12):903-911. 49. Mao J, Maye P, Kogerman P, Tejedor F J, Toftgard R et al. (2002) Regulation of Gli1 Transcriptional Activity in the Nucleus by Dyrk1. J Biol Chem 277(38):35156-35161.
50. Katoh M (2007) Networking of WNT, FGF, Notch, BMP, and Hedgehog Signaling Pathways during Carcinogenesis. Stem Cell Reviews and Reports. 3(1):30-38.
51. Jason K. Sicklick, Yin-Xiong Li, Aruna Jayaraman, Rajesh Kannangai, Yi Qi, Perumal Vivekanandan et al. (2006) Dysregulation of the Hedgehog pathway in human hepatocarcinogenesis. Carcinogenesis vol. 27 no. 4 pp. 748-757.
52. Das S, Harris L G, Metge B J, Liu S, Riker A I, et al. (2009) The hedgehog pathway transcription factor GLI1 promotes malignant behavior of cancer cells by up-regulating osteopontin. J. Biol. Chem., 284(34), 22888-97.
53. Iu'ri D. Louro, Evans C. Bailey, Xingnan Li, Lindsey S. South, Peggy R. McKie-Bell et al. (2002) Comparative Gene Expression Profile Analysis of GLI and c-MYC in an Epithelial Model of Malignant Transformation. CANCER RESEARCH; 62:5867-5873
54. Sandra H. Bigner, Henry S. Friedman, Bert Vogelstein, V V. Jerry Oakes, Darell D. Bigner (1990) Amplification of the c-myc Gene in Human Medulloblastoma Cell Lines and Xenografts Cancer Research; 50:2347-2350.
55. Liu S, Dontu G, Mantle I D, Patel S, Ahn, N.-shik, Jackson, K. W. et al. (2006) Hedgehog signaling and Bmi-1 regulate self-renewal of normal and malignant human mammary stem cells. Cancer Res; 66(12):6063-71. 56. He J, Sheng T, Stelter A A, Li C, Zhang X, Sinha M, et al. (2006) Suppressing Wnt signaling by the hedgehog pathway through sFRP-1. Jour Biol Chem 281(47):35598-35602.
57. David J. Elzi, Meihua Song, Kevin Hakala, Susan T. Weintraub, Yuzuru Shiio (2012) Wnt antagonist SFRP1 functions as secreted mediator of senescence. Mol. Cell. Biol. MCB.06023-11.
58. Buttitta L, Mo R, Hui C C and Fan C M (2003). Interplays of Gli2 and Gli3 and their requirement in mediating Shh-dependent sclerotome induction. Development (Cambridge, England), 130(25):6233-43. 59. Atan Gross, James M. McDonnell,Stanley J. Korsmeyer (1999) BCL-2 family members and the mitochondria in apoptosis. Genes & Dev. 13: 1899-1911.
60. Lawrence Lum, Chi Zhang, Sekyung Oh, Randall K. Mann, Doris P. von Kessler et al. (2003) Hedgehog Signal Transduction via Smoothened Association with a Cytoplasmic Complex Scaffolded by the Atypical Kinesin, Costal-2. Molecular Cell; 12:1261-1274.
61. Cheng-Jeng Tai, Chun-Chao Chang, Ming-Chung Jiang, Chung-Min Yeh, Tzu-Cheng Su et. al. (2012) Clinical-Pathological Correlation Of K-Ras Mutation and Erk Phosphorylation In Colorectal Cancer. Pol J Pathol; 2: 93-100.

1. Novel combinatorial oncoproteins comprising SMO, hFU, ULK3, RAS and ERK12 as potential drug targets in the Hedgehog pathway to control or treat cancer.

2. The combinatorial oncoproteins according to claim 1, wherein the cancer is colon or pancreatic cancer.

3. The combinatorial oncoproteins according to claim 1, wherein the combinatorial oncoproteins are selected from SMO, hFU, ULK3, RAS as potential drug targets in the Hedgehog pathway to control or treat colon cancer.

4. The combinatorial oncoproteins according to claim 1, wherein the combinatorial oncoproteins are selected from SMO, hFU, ULK3, RAS and ERK12 as potential drug targets in the hedgehog pathway to control or treat pancreatic cancer.

5. An in silico method to identify novel combinatorial oncoproteins as claimed in claim 1, as potential drug targets that inhibit hedgehog pathway activity in various cancer cell lines required to control or treat cancer in a subject comprising;
(i) reconstructing a novel hedgehog pathway by collating proteins from the various databases; and
(ii) simulating the logical models of the Hedgehog pathway for normal scenario as well as for cancer scenario in Cell Net Analyzer to identify the proteins as drug targets involved in the abnormal activation of hedgehog pathway in the development of cancer.

6. The method according to claim 5, wherein the logical analysis of step (ii) comprises;
(i) comparing computationally the number of upstream activator proteins DHH, SHH, IHH, PTCH1, SMO, HIP1, STK36, GLI1, GLI2, GLI3R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7A, 8A and 9A; number of downstream activated proteins of DISPATCHED, HHAT, BPM_RUNX3, SHH, DHH, IHH, PTCH1, PTCH2, SMO, HIP1, CDO, BOC, GAS1, ULK3, RAS, STK36, hFU, SUFU, TWIST, PKA_A, BTRCP, CKI_A, GSK3, NUC_GLI1, NUC_GLI2, CYCLIN_B, FOXM1, CYCLIN_D, CYCLIN_E, SNAI1, JAGGED2BCL2, CYCLIN_D2 selected from FIGS. 7C, 8C, and 9C; number of upstream inhibitor proteins of DHH, SHH, IHH, PTCH1, SMO, HIP1, STK36, GLI1, GLI2, GLI3_R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7B, 8B and 9B; and number of downstream inhibited proteins of DISPATCHED, HHAT, BPM_RUNX3, SHH, DHH, IHH, PTCH1, PTCH2, SMO, HIP1, CDO, BOC, GAS1, ULK3, RAS, STK36, hFU, SUFU, TWIST, PKA_A, BTRCP, CKI_A, GSK3, NOTCH1, GLI3R, SKI, NCOR, HDAC, SNO, SIN3A, NUMB, ITCH, NUC_SUFU selected from FIGS. 7D, 8D and 9D of the cancer scenario with each protein of the normal scenario;
(ii) identifying the proteins with significant variations in cancer scenario with respect to the normal scenario; and
(iii) selecting combinations of target proteins from step (ii) for various cancer scenario and perturbing said combination of proteins in the treatment scenario and thereby inhibiting the expression of the output oncoproteins of the hedgehog pathway causing cancer.

7. The method according to claim 6, wherein the number of upstream activator proteins in the cancer scenario is greater than that of the normal scenario thereby effecting the expression of the output oncoproteins.

8. The method according to claim 6, wherein each target protein is assigned ‘0’ or ‘OFF’ and ‘1’ or ‘ON’ to up regulate or down regulate the expression of said protein.

9. The method according to claim 6, wherein the output oncoproteins comprises JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA.

10. The method according to claim 9, wherein the down regulation of output oncoproteins alters the phenotypic outcomes or cellular responses such as Cell proliferation, Cell cycle progression and Endothelial to Mesenchymal transition and also down regulates the pathways such as WNT, NOTCH and Anti-Apoptosis.

11. The method according to claim 5, wherein the combinatorial oncoproteins as potential drug targets comprises (a) combination of SMO, hFU, ULK3 and RAS; and (b) combination of SMO, hFU, ULK3, RAS and ERK12.

12. The method according to claim 5, wherein the type of cancer treated is selected from pancreatic or colon cancer.

13. The method according to claim 5, wherein the databases is selected from KEGG, PATHWAY CENTRAL, BIOCARTA, PROTEIN LOUNGE, NETPATH, GENEGO and other relevant databases.

14. The method according to claim 5, wherein the hedgehog pathway comprises 57 proteins of which 52 are core proteins, 5 cross talk protein molecules obtained from other pathways, 6 cellular or phenotypic expressions and 96 hyper-interactions.

15. An in silico method for selecting cancer treatment regime for colon cancer using novel combinatorial oncoproteins as claimed in claim 3, further comprising perturbation of logical states of combination proteins selected from SMO, hFU, ULK3 and RAS from 1 (“ON”) to 0 (“OFF”) of the hedgehog pathway in the treatment scenario to down regulate the expression of GLI transcription factors and subsequently suppressing the expression of output onco proteins such as JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA as well as the phenotypic expressions of the colon cancer cell line.

16. An in silico method for selecting cancer treatment regime for pancreatic cancer using novel combinatorial oncoproteins as claimed in claim 4, further comprising perturbation of the combination proteins selected from SMO, hFU, ULK3, RAS and ERK12 from 1 (“ON”) to 0 (“OFF”) of the hedgehog pathway in the treatment scenario to down regulate the expression of GLI transcription factors and subsequently suppressing the output proteins such as JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA as well as the phenotypic expressions of the pancreatic cancer cell line.

17. Novel combinatorial oncoproteins comprising SMO, hFU, ULK3, RAS and ERK12 as potential drug targets in the Hedgehog pathway to control or treat cancer.

18. Combinatorial oncoproteins according to claim 17, wherein the cancer is colon or pancreatic cancer.

19. Combinatorial oncoproteins according to claim 17, wherein the combinatorial oncoproteins are selected from SMO, hFU, ULK3, RAS as potential drug targets in the Hedgehog pathway to control or treat colon cancer.

20. Combinatorial oncoproteins according to claim 17, wherein the combinatorial oncoproteins are selected from SMO, hFU, ULK3, RAS and ERK12 as potential drug targets in the hedgehog pathway to control or treat pancreatic cancer.

21. Novel combinatorial oncoproteins biomarkers for the oncoproteins as claimed in claim 3, wherein the biomarkers enable identification of target oncoproteins of the hedgehog pathway for control or treat colon.

22. The novel combinatorial oncoproteins biomarkers according to claim 21, wherein the cancer is colon or pancreatic cancer.

23. The novel combinatorial oncoproteins biomarkers according to claim 21, wherein the combinatorial oncoproteins biomarkers are selected from SMO, hFU, ULK3, RAS as potential drug targets in the Hedgehog pathway to control or treat colon cancer.

24. The novel combinatorial oncoproteins biomarkers according to claim 21, wherein the combinatorial oncoproteins biomarkers are selected from SMO, hFU, ULK3, RAS and ERK12 as potential drug targets in the hedgehog pathway to control or treat pancreatic cancer.

25. An in silico method to identify novel combinatorial oncoproteins biomarkers for novel combinatorial oncoproteins as claimed in claim 1, as potential drug targets that inhibit hedgehog pathway activity in various cancer cell lines required to control or treat cancer in a subject comprising;
(i) reconstructing a novel hedgehog pathway by collating proteins from the various databases; and
(ii) simulating the logical models of the Hedgehog pathway for normal scenario as well as for cancer scenario in Cell Net Analyzer to identify the proteins as drug targets involved in the abnormal activation of hedgehog pathway in the development of cancer.

26. The method according to claim 25, wherein the logical analysis of step (ii) comprises;
(i) comparing computationally the number of upstream activator proteins DHH, SHH, IHH, PTCH1, SMO, HIP1, STK36, GLI1, GLI2, GLI3R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7A, 8A and 9A; number of downstream activated proteins of DISPATCHED, HHAT, BPM_RUNX3, SHH, DHH, IHH, PTCH1, PTCH2, SMO, HIP1, CDO, BOC, GAS1, ULK3, RAS, STK36, hFU, SUFU, TWIST, PKA_A, BTRCP, CKI_A, GSK3, NUC_GLI1, NUC_GLI2, CYCLIN_B, FOXM1, CYCLIN_D, CYCLIN_E, SNAI1, JAGGED2BCL2, CYCLIN_D2 selected from FIGS. 7C, 8C, and 9C; number of upstream inhibitor proteins of DHH, SHH, IHH, PTCH1, SMO, HIP1, STK36, GLI1, GLI2, GLI3R, NUC_GLI1, NUC_GLI2, CTNNB_TCF4, FOXM1, PDGFRA, BMI, OPN, CYCLIN_D, CYCLIN_E, CMYC, SNAI1, JAGGED2, SFRP, WNT, BCL2, CYCLIN_D2 selected from FIGS. 7B, 8B and 9B; and number of downstream inhibited proteins of DISPATCHED, HHAT, BPM_RUNX3, SHH, DHH, IHH, PTCH1, PTCH2, SMO, HIP1, CDO, BOC, GAS1, ULK3, RAS, STK36, hFU, SUFU, TWIST, PKA_A, BTRCP, CKI_A, GSK3, NOTCH1, GLI3R, SKI, NCOR, HDAC, SNO, SIN3A, NUMB, ITCH, NUC_SUFU selected from FIGS. 7D, 8D and 9D of the cancer scenario with each protein of the normal scenario;
(ii) identifying the proteins with significant variations in cancer scenario with respect to the normal scenario; and
(iii) selecting combinations of target proteins from step (ii) for various cancer scenario and perturbing said combination of proteins in the treatment scenario and thereby inhibiting the expression of the output oncoproteins of the hedgehog pathway causing cancer.

27. The method according to claim 26, wherein the number of upstream activator proteins in the cancer scenario is greater than that of the normal scenario thereby effecting the expression of the output oncoproteins.

28. The method according to claim 26, wherein each target protein is assigned ‘O’ or ‘OFF’ and ‘1’ or ‘ON’ to up regulate or down regulate the expression of said protein.

29. The method according to claim 26, wherein the output oncoproteins comprises JAGGED2, WNT, SFRP, CYCLIN_B, CYCLIN_D, CYCLIN_D2, CYCLIN_E, OPN, SNAI1, CMYC, BMI, BCL2, FOXM1 and PDGFRA.

30. The method according to claim 29, wherein the down regulation of output oncoproteins alters the phenotypic outcomes or cellular responses such as Cell proliferation, Cell cycle progression and Endothelial to Mesenchymal transition and also down regulates the pathways such as WNT, NOTCH and Anti-Apoptosis.

31. The method according to claim 25, wherein the combinatorial oncoproteins biomarkers as potential drug targets comprises (a) combination of SMO, hFU, ULK3 and RAS; and (b) combination of SMO, hFU, ULK3, RAS and ERK12.

32. The method according to claim 25, wherein the type of cancer treated is selected from pancreatic or colon cancer.

33. The method according to claim 25, wherein the databases is selected from KEGG, PATHWAY CENTRAL, BIOCARTA, PROTEIN LOUNGE, NETPATH, GENEGO and other relevant databases.

34. The method according to claim 25, wherein the hedgehog pathway comprises 57 proteins of which 52 are core proteins, 5 cross talk protein molecules obtained from other pathways, 6 cellular or phenotypic expressions and 96 hyper-interactions.
